(19) United States
(12) Patent Application Publication
(10) Pub. No.: US 2023/0240656 A1
(43) Pub. Date: Aug. 3, 2023

(54) ADAPTABLE USER INTERFACE FOR A MEDICAL IMAGING SYSTEM

(71) Applicant: KONINKLIJKE PHILIPS N.V., EINDHOVEN (NL)

(72) Inventors: Anup Agarwal, Mill Creek, WA (US); Carina Pereira, Bothell, WA (US); Stefan Burton Roger, Duvall, WA (US); Yang Jiang, Bothell, WA (US); Earl M. Canfield, II, New Braunfels, TX (US); Shannon Renee Fox, Everett, WA (US)

(21) Appl. No.: 18/011,020

(22) PCT Filed: Jun. 17, 2021
(86) PCT No.: PCT/EP2021/066325

§ 371 Date: Dec. 16, 2022

Related U.S. Application Data
(60) Provisional application No. 63/043,822, filed on Jun. 25, 2020.

(51) Int. Cl. A61B 8/00; G16H 40/60; G06V 20/50; G06V 10/82; G06V 10/44; A61B 8/08

(52) U.S. Cl. CPC A61B 8/467; G16H 40/60; G06V 20/50; G06V 10/82; G06V 10/44; A61B 8/54; A61B 8/5223; A61B 8/464; G06V 2201/031

(57) ABSTRACT

An ultrasound imaging system may include a user interface that may be adapted based, at least in part, on usage data of one or more users. The functions of soft or hard controls may be changed in some examples. The layout or appearance of soft or hard controls may be altered in some examples. In some examples, seldom used controls may be removed from the user interface. In some examples, the user interface may be adapted based on an anatomical feature being imaged. In some examples, the usage data may be analyzed by an artificial intelligence/machine learning model, which may provide outputs that may be used to adapt the user interface.

### TECHNICAL FIELD

The present disclosure pertains to adaptable user interfaces of a medical imaging system such as an ultrasound imaging systems, for example a user interface that adapts automatically based on prior usage of the user interface.

### BACKGROUND

User interfaces (UI), particularly graphical user interfaces, are a critical aspect of the overall user experience for any operator of a medical imaging system, such as ultrasound imaging systems. Typically, users operate the medical imaging system in a specific way, which can vary between users based on several factors, for example, personal preference (e.g., follows or doesn't follow standard protocols, heavy time gain compensation user), geography, user type (e.g., physician, sonographer), and application (e.g., abdominal, vascular, breast). However, few, if any, current medical imaging systems on the market permit customization of the UI by the user, and none have UIs that adapt over time.

### SUMMARY

Systems and methods are disclosed that may overcome the limitations of current medical imaging system user interfaces by dynamically modifying (e.g., adapting, adjusting) the presentation of hard and/or soft controls based, at least in part, upon analysis of prior button usage, keystrokes, and/or control-sequencing patterns of one or more users (collectively, usage data). In some applications, the flow of ultrasound procedures may be simplified and more efficient for users over prior art fixed user interface (UI) systems.

As disclosed herein, a UI for a medical imaging system may include a dynamic button layout that allows a user the ability to customize button location as well to show/hide buttons and on what page buttons will appear. As disclosed herein, a processor or processors may analyze usage data, for example usage data stored in log files including logs of prior keystrokes and/or sequences of control selections, to determine a percentage usage of particular controls (e.g., buttons) and/or typical order of control usage. In some examples, the processor or processors may implement an artificial intelligence, machine learning, and/or deep learning model that has been trained, for example on previously-obtained log files, to analyze usage data (e.g., keystroke and control-sequencing patterns entered by users or user types for a given procedure). Based on the analysis, the processor or processors may adjust the UI dynamic button layout based on the output of the trained model.

According to at least one example of the present disclosure, a medical imaging system may include a user interface comprising a plurality of controls, each of the plurality of controls configured to be manipulated by a user for changing an operation of the medical imaging system, a memory configured to store usage data resulting from the manipulation of the plurality of controls, and a processor in communication with the user interface and the memory, wherein the processor is configured to receive the usage data, determine, based on the usage data, a first control of the plurality of controls associated with lower frequency of usage than a second control of the plurality of controls, and adapt the user interface based on the frequency of usage by reducing a visibility of the first control, increasing the visibility of the second control, or a combination thereof.

According to at least one example of the present disclosure, a medical imaging system may include a user interface comprising a plurality of controls configured to be manipulated by a user for changing an operation of the medical imaging system, a memory configured to store usage data resulting from the manipulation of the plurality of controls, and a processor in communication with the user interface and the memory, the processor configured to receive the usage data, receive an indication of a first selected control of the plurality of controls, wherein the first selected control is associated with a first function, determine, based at least in part on the usage data and the first function, a next predicted function, and following manipulation of the first control, adapt the user interface by changing the function of one of the plurality of controls to the next predicted function, increasing a visibility of the control configured to perform the next predicted function relative to other controls of the plurality of controls, or a combination thereof.

### BRIEF DESCRIPTION OF THE DRAWINGS

FIG. 1 is a block diagram of an ultrasound system in accordance with principles of the present disclosure.

FIG. 2 is a block diagram illustrating an example processor in accordance with principles of the present disclosure.

FIG. 3 is an illustration of a portion of an ultrasound imaging system in accordance with examples of the present disclosure.

FIG. 4 is an illustration of soft controls provided on a display according to examples of the present disclosure.

FIG. 5A is an illustration of soft controls provided on a display according to examples of the present disclosure.

FIG. 5B is an illustration of the soft controls provided on the display according to examples of the present disclosure.

FIG. 6 is an illustration of soft controls provided on a display according to examples of the present disclosure.

FIG. 7 is an illustration of soft controls provided on a display according to examples of the present disclosure.

FIG. 8A is an illustration of soft controls provided on a display according to examples of the present disclosure.

FIG. 8B is an illustration of soft controls provided on a display according to examples of the present disclosure.

FIG. 9 is an illustration of example ultrasound images on a display and soft controls provided on a display according to examples of the present disclosure.

FIG. 10 is a graphical depiction of an example of statistical analysis of one or more log files in accordance with examples of the present disclosure.

FIG. 11 is a graphical depiction of an example of statistical analysis of one or more log files in accordance with examples of the present disclosure.

FIG. 12 is an illustration of a neural network that may be used to analyze usage data in accordance with examples of the present disclosure.

FIG. 13 is an illustration of a cell of a long short term memory model that may be used to analyze usage data in accordance with examples of the present disclosure.

FIG. 14 is a block diagram of a process for training and deployment of a neural network in accordance with the principles of the present disclosure.

FIG. 15 shows a graphical overview of a user moving a button within a page of a menu provided on a display in accordance with examples of the present disclosure.

FIG. 16 shows a graphical overview of a user moving a button between pages of a menu on a display in accordance with examples of the present disclosure.

FIG. 17 shows a graphical overview of a user swapping locations of buttons on a display in accordance with examples of the present disclosure.

FIG. 18 shows a graphical overview of a user moving a group of buttons on a display in accordance with examples of the present disclosure.

FIG. 19 shows a graphical overview of a user changing a rotary control to a list button on a display in accordance with examples of the present disclosure.

### DETAILED DESCRIPTION

The following description of certain embodiments is merely exemplary in nature and is in no way intended to limit the invention or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the present system. Moreover, for the purpose of clarity, detailed descriptions of certain features will not be discussed when they would be apparent to those with skill in the art so as not to obscure the description of the present system. The following detailed description is therefore not to be taken in a limiting sense, and the scope of the present system is defined only by the appended claims.

Medical imaging system users have expressed frustration at the inability to customize user interfaces (UI) of the medical imaging system. Although different users may vary significantly from each other in how they operate a medical imaging system, each user typically follows the same or similar pattern each time they use the medical imaging system, particularly for a same application (e.g., a fetal scan, echocardiogram in ultrasound imaging). That is, for a particular application, a user typically uses the same set of controls, performs the same tasks, and/or performs the same tasks in the same order each time. This is especially true when users are following imaging technician-driven workflow where the imaging technician (e.g., a sonographer) performs the imaging examination which is read at a later time by a reviewing physician (e.g., radiologist). This workflow-based exam is common in North America.

Users often adjust, or customize, system settings in order to optimize their workflow-based exam. Such customizations can improve the efficiency and quality of the exam for that particular user. Customizations, though, can be time-consuming and/or may be required to be re-entered each time the user initializes a particular system. The inventors have thus recognized that, in addition to, or as an alternative to, permitting users to perform their own customizations of the UI, the medical imaging system may be arranged to “learn” the preferences of the user and automatically adapt the UI to the user's preferences without the user being required to perform the customizations manually. Thus, substantial time and effort may be saved, and quality of the exam may be enhanced.

As disclosed herein, a medical imaging system may analyze and automatically adapt (e.g., adjust, change) the UI of the ultrasound imaging system based, at least in part, on usage data (e.g., keystrokes, patterns of button pushes) collected from one or more users of the medical imaging system. In some examples, the medical imaging system may fade less-used controls on a display. In some examples, the degree of fading may increase overtime until the controls are removed from the display. In some examples, less-used controls may be moved further down on a display and/or moved to a second or subsequent page of a menu of the UI. In some examples, highly used controls may be highlighted (e.g., appear brighter or in a different color than other controls). In some examples, the medical imaging system may infer which control the user will select next and highlight the control on the display and/or control panel. In some examples, the medical imaging system will alter the functionality of a soft control (e.g., button on a touch screen) or a hard control (e.g., switch, dial, slider) based on an inference of what control function the user will use next. In some examples, this analysis and adaptation may be provided for each individual user of the medical imaging system. Thus, the medical imaging system may provide a customized, adaptable UI for each user without requiring user manipulation of the system settings. In some applications, automatically adapting the UI may reduce exam time, improve efficiency, and/or provide ergonomic benefits to the user.

The examples disclosed herein are provided in reference to ultrasound imaging systems. However, this is for illustrative purposes only and the adaptable UI and features thereof disclosed herein may be applied to other medical imaging systems.

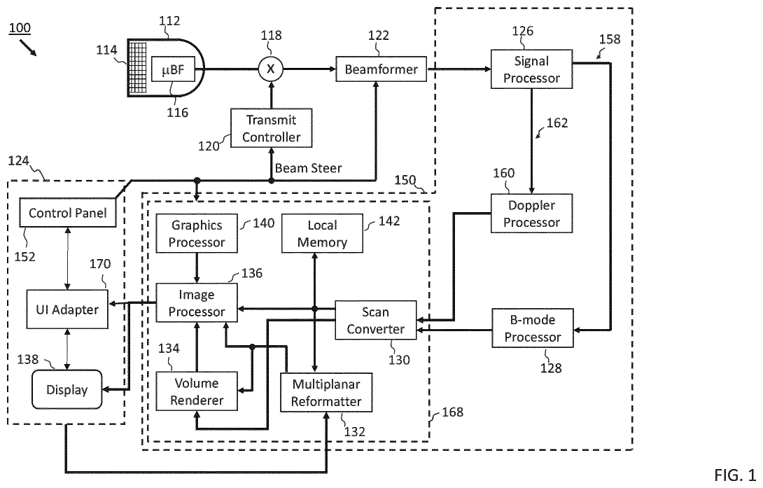

FIG. 1 shows a block diagram of an ultrasound imaging system 100 constructed in accordance with the principles of the present disclosure. An ultrasound imaging system 100 according to the present disclosure may include a transducer array 114, which may be included in an ultrasound probe 112, for example an external probe or an internal probe such as an Intra Cardiac Echography (ICE) probe or a Trans Esophagus Echography (TEE) probe. In other embodiments, the transducer array 114 may be in the form of a flexible array configured to be conformably applied to a surface of subject to be imaged (e.g., patient). The transducer array 114 is configured to transmit ultrasound signals (e.g., beams, waves) and receive echoes responsive to the ultrasound signals. A variety of transducer arrays may be used, e.g., linear arrays, curved arrays, or phased arrays. The transducer array 114, for example, can include a two dimensional array (as shown) of transducer elements capable of scanning in both elevation and azimuth dimensions for 2D and/or 3D imaging. As is generally known, the axial direction is the direction normal to the face of the array (in the case of a curved array the axial directions fan out), the azimuthal direction is defined generally by the longitudinal dimension of the array, and the elevation direction is transverse to the azimuthal direction.

In some embodiments, the transducer array 114 may be coupled to a microbeamformer 116, which may be located in the ultrasound probe 112, and which may control the transmission and reception of signals by the transducer elements in the array 114. In some embodiments, the microbeamformer 116 may control the transmission and reception of signals by active elements in the array 114 (e.g., an active subset of elements of the array that define the active aperture at any given time).

In some embodiments, the microbeamformer 116 may be coupled, e.g., by a probe cable or wirelessly, to a transmit/receive (T/R) switch 118, which switches between transmission and reception and protects the main beamformer 122 from high energy transmit signals. In some embodiments, for example in portable ultrasound systems, the T/R switch 118 and other elements in the system can be included in the ultrasound probe 112 rather than in the ultrasound system base, which may house the image processing electronics. An ultrasound system base typically includes software and hardware components including circuitry for signal processing and image data generation as well as executable instructions for providing a user interface (e.g., processing circuitry 150 and user interface 124).

The transmission of ultrasonic signals from the transducer array 114 under control of the microbeamformer 116 is directed by the transmit controller 120, which may be coupled to the T/R switch 218 and a main beamformer 122. The transmit controller 120 may control the direction in which beams are steered. Beams may be steered straight ahead from (orthogonal to) the transducer array 114, or at different angles for a wider field of view. The transmit controller 120 may also be coupled to a user interface 124 and receive input from the user's operation of a user control. The user interface 124 may include one or more input devices such as a control panel 152, which may include one or more mechanical controls (e.g., buttons, encoders, etc.), touch sensitive controls (e.g., a trackpad, a touchscreen, or the like), and/or other known input devices.

In some embodiments, the partially beamformed signals produced by the microbeamformer 116 may be coupled to a main beamformer 122 where partially beamformed signals from individual patches of transducer elements may be combined into a fully beamformed signal. In some embodiments, microbeamformer 116 is omitted, and the transducer array 114 is under the control of the main beamformer 122 which performs all beamforming of signals. In embodiments with and without the microbeamformer 116, the beamformed signals of the main beamformer 122 are coupled to processing circuitry 150, which may include one or more processors (e.g., a signal processor 126, a B-mode processor 128, a Doppler processor 160, and one or more image generation and processing components 168) configured to produce an ultrasound image from the beamformed signals (e.g., beamformed RF data).

The signal processor 126 may be configured to process the received beamformed RF data in various ways, such as bandpass filtering, decimation, I and Q component separation, and harmonic signal separation. The signal processor 126 may also perform additional signal enhancement such as speckle reduction, signal compounding, and noise elimination. The processed signals (also referred to as I and Q components or IQ signals) may be coupled to additional downstream signal processing circuits for image generation. The IQ signals may be coupled to a plurality of signal paths within the system, each of which may be associated with a specific arrangement of signal processing components suitable for generating different types of image data (e.g., B-mode image data, Doppler image data). For example, the system may include a B-mode signal path 158 which couples the signals from the signal processor 126 to a B-mode processor 128 for producing B-mode image data.

The B-mode processor can employ amplitude detection for the imaging of structures in the body. The signals produced by the B-mode processor 128 may be coupled to a scan converter 130 and/or a multiplanar reformatter 132. The scan converter 130 may be configured to arrange the echo signals from the spatial relationship in which they were received to a desired image format. For instance, the scan converter 130 may arrange the echo signal into a two dimensional (2D) sector-shaped format, or a pyramidal or otherwise shaped three dimensional (3D) format. The multiplanar reformatter 132 can convert echoes which are received from points in a common plane in a volumetric region of the body into an ultrasonic image (e.g., a B-mode image) of that plane, for example as described in U.S. Pat. No. 6,443,896 (Detmer). The scan converter 130 and multiplanar reformatter 132 may be implemented as one or more processors in some embodiments.

A volume renderer 134 may generate an image (also referred to as a projection, render, or rendering) of the 3D dataset as viewed from a given reference point, e.g., as described in U.S. Pat. No. 6,530,885 (Entrekin et al.). The volume renderer 134 may be implemented as one or more processors in some embodiments. The volume renderer 134 may generate a render, such as a positive render or a negative render, by any known or future known technique such as surface rendering and maximum intensity rendering.

In some embodiments, the system may include a Doppler signal path 162 which couples the output from the signal processor 126 to a Doppler processor 160. The Doppler processor 160 may be configured to estimate the Doppler shift and generate Doppler image data. The Doppler image data may include color data which is then overlaid with B-mode (i.e. grayscale) image data for display. The Doppler processor 160 may be configured to filter out unwanted signals (i.e., noise or clutter associated with non-moving tissue), for example using a wall filter. The Doppler processor 160 may be further configured to estimate velocity and power in accordance with known techniques. For example, the Doppler processor may include a Doppler estimator such as an auto-correlator, in which velocity (Doppler frequency) estimation is based on the argument of the lag-one autocorrelation function and Doppler power estimation is based on the magnitude of the lag-zero autocorrelation function. Motion can also be estimated by known phase-domain (for example, parametric frequency estimators such as MUSIC, ESPRIT, etc.) or time-domain (for example, cross-correlation) signal processing techniques. Other estimators related to the temporal or spatial distributions of velocity such as estimators of acceleration or temporal and/or spatial velocity derivatives can be used instead of or in addition to velocity estimators. In some embodiments, the velocity and/or power estimates may undergo further threshold detection to further reduce noise, as well as segmentation and post-processing such as filling and smoothing. The velocity and/or power estimates may then be mapped to a desired range of display colors in accordance with a color map. The color data, also referred to as Doppler image data, may then be coupled to the scan converter 230, where the Doppler image data may be converted to the desired image format and overlaid on the B-mode image of the tissue structure to form a color Doppler or a power Doppler image. In some examples, the scan converter 130 may align the Doppler image and B-mode image

Outputs from the scan converter 130, the multiplanar reformatter 132, and/or the volume renderer 134 may be coupled to an image processor 136 for further enhancement, buffering and temporary storage before being displayed on an image display 138. A graphics processor 140 may generate graphic overlays for display with the images. These graphic overlays can contain, e.g., standard identifying information such as patient name, date and time of the image, imaging parameters, and the like. For these purposes the graphics processor may be configured to receive input from the user interface 124, such as a typed patient name or other annotations. The user interface 124 can also be coupled to the multiplanar reformatter 132 for selection and control of a display of multiple multiplanar reformatted (MPR) images.

The ultrasound imaging system 100 may include local memory 142. Local memory 142 may be implemented as any suitable non-transitory computer readable medium (e.g., flash drive, disk drive). Local memory 142 may store data generated by the ultrasound imaging system 100 including ultrasound images, log files including usage data, executable instructions, imaging parameters, training data sets, and/or any other information necessary for the operation of the ultrasound imaging system 100. Although not all connections are shown to avoid obfuscation of FIG. 1, local memory 142 may be accessible by additional components other than the scan converter 130, multiplanar reformatter 132, and image processor 136. For example, the local memory 142 may be accessible to the graphics processor 140, transmit controller 120, signal processor 126, user interface 124, etc.

As mentioned previously ultrasound imaging system 100 includes user interface 124. User interface 124 may include display 138 and control panel 152. The display 138 may include a display device implemented using a variety of known display technologies, such as LCD, LED, OLED, or plasma display technology. In some embodiments, display 138 may comprise multiple displays. The control panel 152 may be configured to receive user inputs (e.g., pre-set number of frames, filter window length, imaging mode). The control panel 152 may include one or more hard controls (e.g., buttons, knobs, dials, encoders, mouse, trackball or others). Hard controls may sometimes be referred to as mechanical controls. In some embodiments, the control panel 152 may additionally or alternatively include soft controls (e.g., GUI control elements, or simply GUI controls such as buttons and sliders) provided on a touch sensitive display. In some embodiments, display 138 may be a touch sensitive display that includes one or more soft controls of the control panel 152.

According to examples of the present disclosure, ultrasound imaging system 100 may include a user interface (UI) adapter 170 that automatically adapts the appearance and/or functionality of the user interface 124 based, at least in part, on usage of the ultrasound imaging system 100 by a user. In some examples, the UI adapter 170 may be implemented by one or more processors and/or application specific integrated circuits. The UI adapter 170 may collect usage data from the user interface 124. Examples of usage data include, but are not limited to, keystrokes, button pushes, other manipulation (e.g., selection) of hard controls (e.g., turning a dial, flipping a switch), screen touches, other manipulation of soft controls, menu selections and navigation, and voice commands. In some examples, additional usage data may be received such as geographical location of the ultrasound machine, type of ultrasound probe used, unique user identifier, type of exam, and/or object imaged by the ultrasound imaging system 100. In some examples, some additional usage data may be provided by a user via the user interface 124, image processor 136, and/or preprogrammed and stored in ultrasound imaging system 100 (e.g., local memory 142).

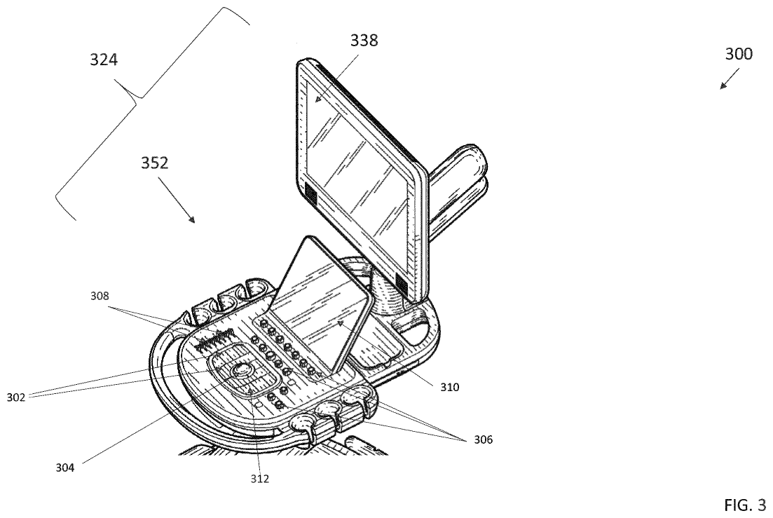

The UI adapter 170 may perform live capture and analysis of the usage data. That is, the UI adapter 170 may receive and analyze the usage data as the user is interacting with the ultrasound imaging system 100 through the user interface 124. In these examples, the UI adapter 170 may automatically adapt the user interface 124 based, at least in part, on the usage data while the user is interacting with the user interface 124. However, in some examples, the UI adapter 170 may automatically adapt the user interface 124 when the user is not interacting with the user interface 124 (e.g., a pause in the workflow, end of exam). Alternatively, or in addition to, live analysis, the UI adapter 170 may capture and store the usage data (e.g., as log files in local memory 142) and analyze the stored usage data at a later time. In some examples when usage data is analyzed later, the UI adapter 170 may automatically adapt the user interface 124, but these adaptations may not be provided to the user until the next time the user interacts with the ultrasound imaging system 100 (e.g., user starts next step in the workflow, next time user logs into ultrasound imaging system 100). Additional details of example adaptions of the user interface 124 the UI adapter 170 may perform are discussed with reference to FIGS. 3-9.

In some examples, the UI adapter 170 may include and/or implement any one or more machine learning models, deep learning models, artificial intelligence algorithms, and/or neural networks which may analyze the usage data and adapt the user interface 124. In some examples, UI adapter 170 may include a long short term (LSTM) model, deep neural network (DNN), a convolutional neural network (CNN), a recurrent neural network (RNN), an autoencoder neural network, or the like, to adapt the control panel 152 and/or display 138. The model and/or neural network may be implemented in hardware (e.g., neurons are represented by physical components) and/or software (e.g., neurons and pathways implemented in a software application) components. The model and/or neural network implemented according to the present disclosure may use a variety of topologies and learning algorithms for training the model and/or neural network to produce the desired output. For example, a software-based neural network may be implemented using a processor (e.g., single or multi-core CPU, a single GPU or GPU cluster, or multiple processors arranged for parallel-processing) configured to execute instructions, which may be stored in computer readable medium, and which when executed cause the processor to perform a trained algorithm for adapting the user interface 124 (e.g., determine most and/or least used controls, predict a next control selected by a user in a sequence, altering an appearance of controls shown on display 138, altering the function of a physical control on control panel 152). In some embodiments, the UI adapter 170 may implement a model and/or neural network in combination with other data processing methods (e.g., statistical analysis).

In various embodiments, the model(s) and/or neural network(s) may be trained using any of a variety of currently known or later developed learning techniques to obtain a model and/or neural network (e.g., a trained algorithm, transfer function, or hardware-based system of nodes) that is configured to analyze input data in the form of screen touches, keystrokes, control manipulations, usage log files, other user input data, ultrasound images, measurements, and/or statistics. In some embodiments, the model and/or neural network may be statically trained. That is, the model and/or neural network may be trained with a data set and deployed on the UI adapter 170. In some embodiments, the model and/or neural network may be dynamically trained. In these embodiments, the model and/or neural network may be trained with an initial data set and deployed on the ultrasound system 100. However, the model and/or neural network may continue to train and be modified based on inputs acquired by the UI adapter 170 after deployment of the model and/or neural network on the UI adapter 170.

Although shown within the user interface 124 in FIG. 1, the UI adapter 170 need not be physically located within or immediately adjacent to the user interface 124. For example, UI adapter 170 may be located with processing circuitry 150.

In some embodiments, various components shown in FIG. 1 may be combined. For instance, in some examples, a single processor may implement multiple components of the processing circuitry 150 (e.g., image processor 136, graphics processor 140) as well as the UI adapter 170. In some embodiments, various components shown in FIG. 1 may be implemented as separate components. For example, signal processor 126 may be implemented as separate signal processors for each imaging mode (e.g., B-mode, Doppler, SWE). In some embodiments, one or more of the various processors shown in FIG. 1 may be implemented by general purpose processors and/or microprocessors configured to perform the specified tasks. In some embodiments, one or more of the various processors may be implemented as application specific circuits. In some embodiments, one or more of the various processors (e.g., image processor 136) may be implemented with one or more graphical processing units (GPU).

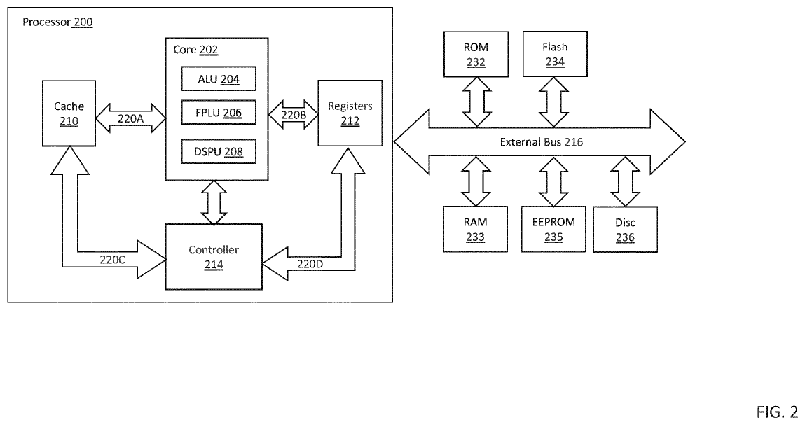

FIG. 2 is a block diagram illustrating an example processor 200 according to principles of the present disclosure. Processor 200 may be used to implement one or more processors and/or controllers described herein, for example, image processor 136, graphics processor 140, and/or UI adapter 170 shown in FIG. 1 and/or any other processor or controller shown in FIG. 1. Processor 200 may be any suitable processor type including, but not limited to, a microprocessor, a microcontroller, a digital signal processor (DSP), a field programmable array (FPGA) where the FPGA has been programmed to form a processor, a graphical processing unit (GPU), an application specific circuit (ASIC) where the ASIC has been designed to form a processor, or a combination thereof.

The processor 200 may include one or more cores 202. The core 202 may include one or more arithmetic logic units (ALU) 204. In some embodiments, the core 202 may include a floating point logic unit (FPLU) 206 and/or a digital signal processing unit (DSPU) 208 in addition to or instead of the ALU 204.

The processor 200 may include one or more registers 212 communicatively coupled to the core 202. The registers 212 may be implemented using dedicated logic gate circuits (e.g., flip-flops) and/or any memory technology. In some embodiments the registers 212 may be implemented using static memory. The register may provide data, instructions and addresses to the core 202.

In some embodiments, processor 200 may include one or more levels of cache memory 210 communicatively coupled to the core 202. The cache memory 210 may provide computer-readable instructions to the core 202 for execution. The cache memory 210 may provide data for processing by the core 202. In some embodiments, the computer-readable instructions may have been provided to the cache memory 210 by a local memory, for example, local memory attached to the external bus 216. The cache memory 210 may be implemented with any suitable cache memory type, for example, metal-oxide semiconductor (MOS) memory such as static random access memory (SRAM), dynamic random access memory (DRAM), and/or any other suitable memory technology.

The processor 200 may include a controller 214, which may control input to the processor 200 from other processors and/or components included in a system (e.g., control panel 152 and scan converter 130 shown in FIG. 1) and/or outputs from the processor 200 to other processors and/or components included in the system (e.g., display 138 and volume renderer 134 shown in FIG. 1). Controller 214 may control the data paths in the ALU 204, FPLU 206 and/or DSPU 208. Controller 214 may be implemented as one or more state machines, data paths and/or dedicated control logic. The gates of controller 214 may be implemented as standalone gates, FPGA, ASIC or any other suitable technology.

The registers 212 and the cache memory 210 may communicate with controller 214 and core 202 via internal connections 220A, 220B, 220C and 220D. Internal connections may implemented as a bus, multiplexor, crossbar switch, and/or any other suitable connection technology.

Inputs and outputs for the processor 200 may be provided via a bus 216, which may include one or more conductive lines. The bus 216 may be communicatively coupled to one or more components of processor 200, for example the controller 214, cache memory 210, and/or register 212. The bus 216 may be coupled to one or more components of the system, such as display 138 and control panel 152 mentioned previously.

The bus 216 may be coupled to one or more external memories. The external memories may include Read Only Memory (ROM) 232. ROM 232 may be a masked ROM, Electronically Programmable Read Only Memory (EPROM) or any other suitable technology. The external memory may include Random Access Memory (RAM) 233. RAM 233 may be a static RAM, battery backed up static RAM, Dynamic RAM (DRAM) or any other suitable technology. The external memory may include Electrically Erasable Programmable Read Only Memory (EEPROM) 235. The external memory may include Flash memory 234. The external memory may include a magnetic storage device such as disc 236. In some embodiments, the external memories may be included in a system, such as ultrasound imaging system 100 shown in FIG. 1, for example local memory 142.

More detailed explanations of examples of adaptations of a UI of an ultrasound imaging system based on usage data from one or more users according to examples of the present disclosure will now be provided.

FIG. 3 is an illustration of a portion of an ultrasound imaging system in accordance with examples of the present disclosure. The ultrasound imaging system 300 may be included in ultrasound imaging system 100 and/or ultrasound imaging system 300 may be used to implement ultrasound imaging system 100. Ultrasound imaging system 300 may include a display 338 and a control panel 352, which may be included as part of a user interface 324 of ultrasound imaging system 300. The display 338 may be used to implement display 138 in some examples and/or control panel 352 may be used to implement control panel 152 in some examples.

The control panel 352 may include one or more hard controls a user may manipulate to operate the ultrasound imaging system 300. In the example shown in FIG. 3, the control panel 352 includes buttons 302, a track ball 304, knobs (e.g., dials) 306, and sliders 308. In some examples, the control panel 352 may include fewer, additional and/or different hard controls, for example, a keyboard, a track pad, and/or rocker switches. The control panel 352 may include a flat panel touch screen 310 in some examples. The touch screen 310 may provide soft controls for the user to manipulate to operate the ultrasound imaging system 300. Examples of soft controls include, but are not limited to, buttons, sliders, and gesture controls (e.g., a two-touch “pinch” motion to zoom out, a one-touch “drag” to draw a line or move a cursor). In some examples, the display 338 may also be a touch screen that provides soft controls. In other examples, touch screen 310 may be omitted, and display 338 may be a touch screen providing soft controls. In some examples, display 338 may not be a touch screen that displays soft controls that may be selected by a user by manipulating one or more hard controls on the control panel 352. For example, the user may manipulate a cursor on display 338 with the track ball 304 and select soft controls on the display 338 by clicking a button 302 when the cursor is over the desired button. The touch screen 310 may optionally be omitted in these examples. In some examples, not shown in FIG. 3, additional hard controls may be provided on a periphery of display 338. In some examples, control panel 352 may include a microphone for accepting verbal commands or inputs from the user. Although not shown in FIG. 3, when ultrasound imaging system 300 is included in a cart-based ultrasound imaging system, user interface 324 may further include a foot pedal that may be manipulated by the user to provide inputs to the ultrasound imaging system 300.

As described herein, in some examples, control panel 352 may include a hard control 312 that has a variable function. That is, the function performed by the hard control 312 is not fixed. In some examples, the function of the hard control 312 is altered by commands executed by a processor of ultrasound imaging system 300, for example, UI adapter 170. The processor may alter the function of hard control 312 based, at least in part, on usage data received by the ultrasound imaging system 300 from a user. Based on an analysis of the usage data, the ultrasound imaging system 300 may predict the next function the user will select. Based on this prediction, the processor may assign the predicted next function to the hard control 312. Optionally, in some examples, the analysis of usage data and prediction may be performed by a different processor than a processor that adapts (e.g., changes) the function of hard control 312. In some examples, the hard control 312 may have an initial function assigned based on an exam type and/or a default setting programmed into the ultrasound imaging system 300. In other examples, the hard control 312 may have no function (e.g., inactive) prior to an initial input from the user. Although hard control 312 is shown as a button in FIG. 3, in other examples, hard control 312 may be a dial, a switch, a slider, and/or any other suitable hard control (e.g., track ball).

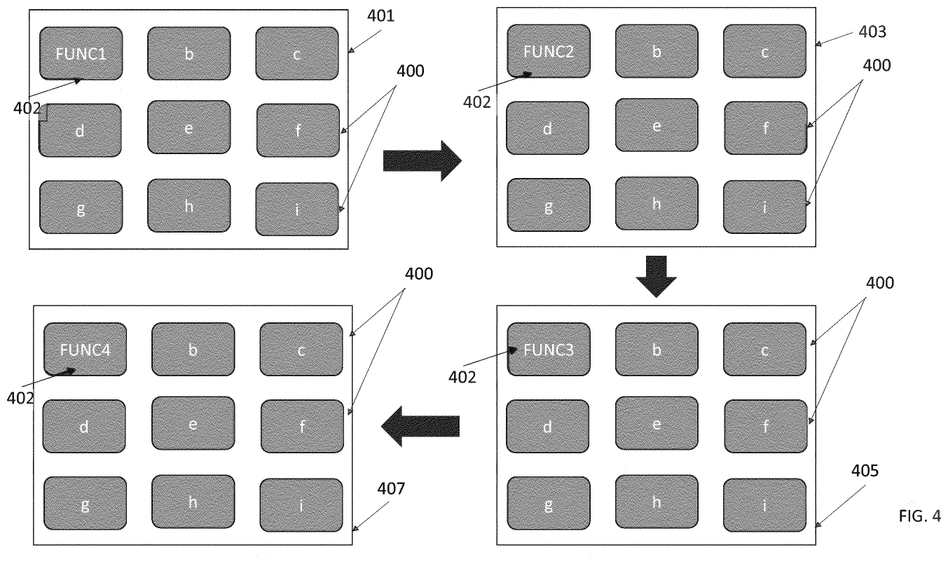

FIG. 4 is an illustration of soft controls provided on a display according to examples of the present disclosure. The soft controls 400 may be provided on a touch screen of an ultrasound imaging system, such as touch screen 310 shown in FIG. 3. The soft controls 400 may also, or alternatively, be provided on a non-touch display, such as display 338 and/or display 138, and a user may interact with the soft controls 400 by manipulating a hard control on a control panel, such as control panel 352 and/or control panel 152. For example, a user may move a cursor over one or more of the soft controls 400 on display 338 with track ball 304 and depress a button 302 to manipulate the soft control 400 to operate the ultrasound imaging system 300. Although all of the soft controls 400 shown in FIG. 4 are buttons, the soft controls 400 may be any combination of soft controls (e.g., buttons, sliders) and any number of soft controls 400 may be provided on the display.

As described with reference to FIG. 3, a control panel may include a hard control (e.g., hard control 312) that has a variable function. Similarly, as described herein, one or more of the soft controls 400 may have a variable function. As shown in panel 401, soft control 402 may initially be assigned a first function FUNC1 (e.g., freeze, acquire). The first function FUNC1 may be based, at least in part, on a particular user that has logged in, a selected exam type, or a default function stored in the ultrasound imaging system (e.g., ultrasound imaging system 100 and/or ultrasound imaging system 300). Alternatively, in some examples, soft control 402 may be disabled at panel 401.

A user may provide an input to the ultrasound imaging system to select a control, such as touching or otherwise manipulating one of the soft controls 400 and/or manipulating a hard control of a control panel (not shown in FIG. 4). In some examples, the user may touch soft control 402. Responsive, at least in part, to the user's input, the function of soft control 402 may be changed to a second function FUNC2 (e.g., acquire, annotate) as shown in panel 403. The second function FUNC2 may be assigned based on a prediction of what the next function the user will select. In some examples, the prediction may be based at least in part on an analysis of usage data. The usage data may include the user input provided in panel 401, and/or may include prior usage data (e.g., earlier user inputs during the exam, user inputs from prior exams). In some examples, the analysis and prediction may be performed by a processor of the ultrasound imaging system, such as UI adapter 170.

A user may provide a second input to the ultrasound imaging system, such as touching or otherwise manipulating one of the soft controls 400 and/or manipulating a hard control of a control panel. In some examples, the user may touch soft control 402, for example, when the processor correctly predicted the next function selected by the user. Responsive, at least in part, to the user's input, the function of soft control 402 may be changed by the processor to a third function FUNC3 (e.g., annotate, calipers) as shown in panel 405. Again, the third function FUNC3 may be assigned based on analysis of usage data. For example, the function assigned as the third function FUNC3 may be different depending on whether the user used the second function FUNC2 (e.g., the processor made a correct prediction) or selected a different function (e.g., the processor made an incorrect prediction).

A user may provide a third input to the ultrasound imaging system, such as touching or otherwise manipulating one of the soft controls 400 and/or manipulating a hard control of a control panel. In some examples, the user may touch soft control 402. Responsive, at least in part, to the user's input, the function of soft control 402 may be changed by the processor to a fourth function FUNC4 (e.g., update, change depth) as shown in panel 407. Again, the fourth function FUNC4 may be assigned based on analysis of usage data. For example, the function assigned as the fourth function FUNC4 may be different depending on whether the processor provided correct predictions at panels 403 and 405. Although changes in functions of soft control 402 are shown for three user inputs, the function of soft control 402 may be altered for any number of user inputs. Furthermore, in some examples, the user inputs provided may be stored for future analysis by the processor for making predictions of the next function desired by the user during a subsequent exam.

By altering the function of one or more hard controls (e.g., hard control 312) and/or one or more soft controls (e.g., soft control 402), the user may keep using the hard or soft control for different functions during an exam. In some applications, this may reduce the user's need to search for a control for a desired function on a user interface (e.g., user interface 124, user interface 324). Reduced searching may reduce time and improve efficiency of the exam. In some applications, using a single hard control for multiple functions may improve ergonomics of an ultrasound imaging system (e.g., ultrasound imaging system 100, ultrasound imaging system 300).

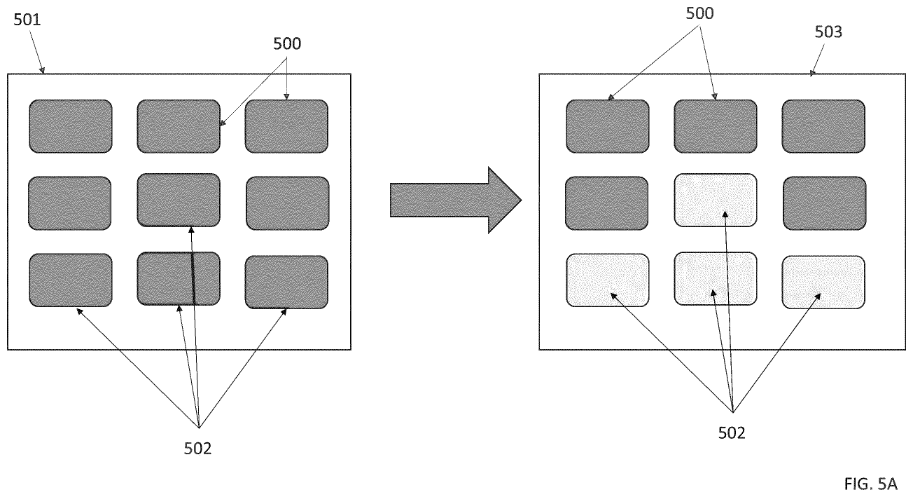

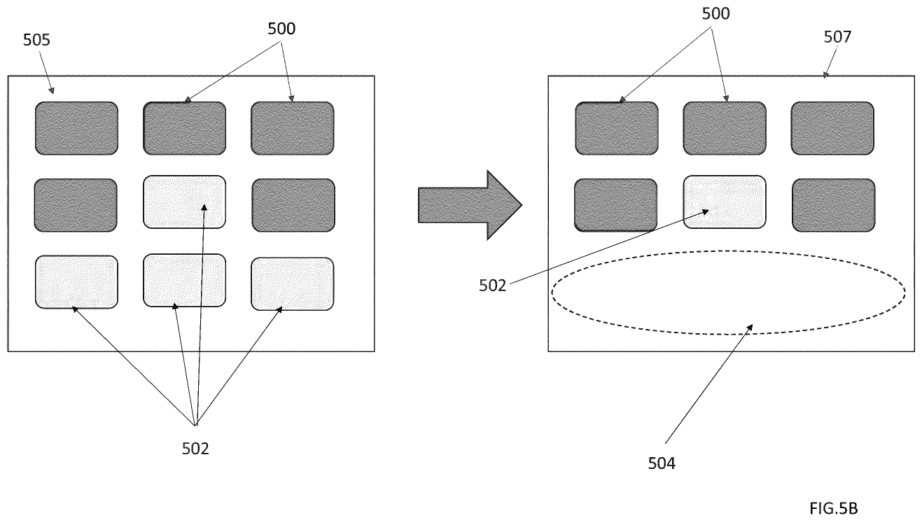

FIG. 5A is an illustration of soft controls provided on a display according to examples of the present disclosure. The soft controls 500 may be provided on a touch screen of an ultrasound imaging system, such as touch screen 310 shown in FIG. 3. The soft controls 500 may also, or alternatively, be provided on a non-touch display, such as display 338 and/or display 138, and a user may interact with the soft controls 500 by manipulating a hard control on a control panel, such as control panel 352 and/or control panel 152. Although all of the soft controls 500 shown in FIG. 5A are buttons, the soft controls 500 may be any combination of soft controls and any number of soft controls 500 may be provided on the display.

In the example shown in FIG. 5A, a processor of the ultrasound imaging system (e.g., UI adapter 170) may analyze usage data to determine which soft controls 500 are used the most by a user. Based on the analysis, the processor may adjust an appearance of the soft controls 500 that are less frequently used. In some examples, such as the one shown in panel 501, all of the soft controls 500 may initially have a same appearance. After a period of use by the user (e.g., one or more user inputs, one or more exams), the appearance of at least one of the soft controls 500 may be altered by the processor. For example, as shown in panel 503, less used soft controls 502 may take on a faded appearance (e.g., dimmer, more translucent relative to other controls) compared to the remaining soft controls 500. Dimming may be achieved by reducing the backlighting of the display at the soft control 502 in some examples. In some examples, the degree of difference in appearance may become more pronounced after further use by the user of the ultrasound system (e.g., additional user inputs, additional exams). For example, the fading may increase over time (e.g., dimness increases, translucency increases).

Optionally, in some examples, as shown in FIG. 5B, one or more of the lesser used soft controls 502 shown in panel 505 may eventually be removed from the display by the processor as shown in panel 507. Removal of soft controls 502 may be based, at least in part, on analysis of the usage data, for example, when the usage data indicates the soft controls 502 are seldom or never selected by the user (e.g., frequency of usage falls below a predetermined threshold value). The time over which soft controls 502 are faded and/or removed may vary based on user preference, frequency of use of the ultrasound imaging system by the user, and/or pre-set settings of the ultrasound imaging system. Although a blank space 504 is shown where the soft controls 502 were removed, in other examples, the processor may replace the removed soft controls 502 with other soft controls, for example, soft controls that the usage data indicates the user selects more frequently.

By altering the appearance of lesser used soft controls, such as by fading, a user's attention may be more easily directed to the most frequently used soft controls. This may reduce the time the user searches for a desired control. By removing unused soft controls, clutter on a display of a UI may be reduced, which may make desired controls easier to find. However, merely altering the appearance of lesser used soft controls may be preferable to some users and/or applications because the layout of the UI is unchanged and the lesser used controls are still available for use.

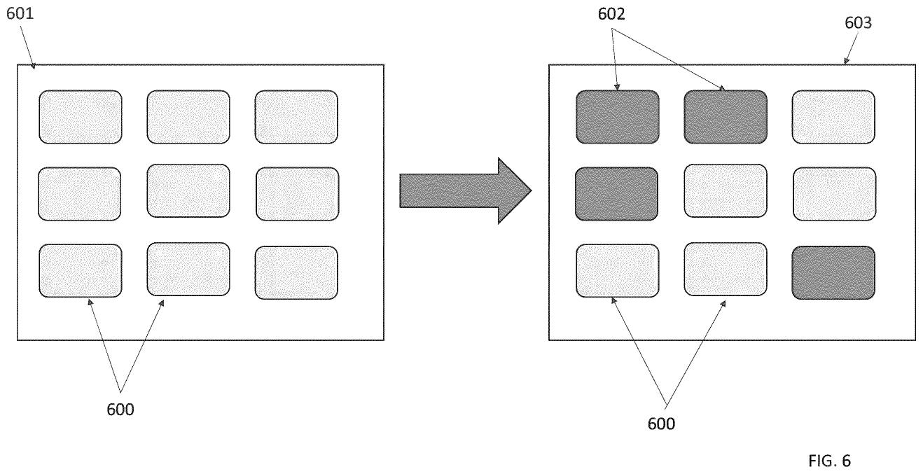

FIG. 6 is an illustration of soft controls provided on a display according to examples of the present disclosure. The soft controls 600 may be provided on a touch screen of an ultrasound imaging system, such as touch screen 310 shown in FIG. 3. The soft controls 600 may also, or alternatively, be provided on a non-touch display, such as display 338 and/or display 138, and a user may interact with the soft controls 600 by manipulating a hard control on a control panel, such as control panel 352 and/or control panel 152. Although all of the soft controls shown in FIG. 6 are buttons, the soft controls 600 may be any combination of soft controls and any number of soft controls 600 may be provided on the display.

In the example shown in FIG. 6, a processor of the ultrasound imaging system (e.g., UI adapter 170) may analyze usage data to determine which soft controls 600 are used the most by a user. Based on the analysis, the processor may adjust an appearance of the soft controls 600 that are most frequently used. In some examples, such as the one shown in panel 601, all of the soft controls 600 may initially have a same appearance. After a period of use by the user (e.g., one or more user inputs, one or more exams), the appearance of at least one of the soft controls 600 may be altered by the processor. For example, as shown in panel 603, more frequently used soft controls 602 may take on a highlighted appearance (e.g., brighter, different color relative to other controls) compared to the remaining soft controls 600. In some examples, the degree of different in appearance may become more pronounced after further use by the user of the ultrasound system (e.g., additional user inputs, additional exams). For example, the highlighting may increase over time (e.g., brightness increases).

By altering the appearance of most frequently used soft controls, such as by highlighting, a user's attention may be more easily directed to the most frequently used soft controls. This may reduce the time the user searches for a desired control.

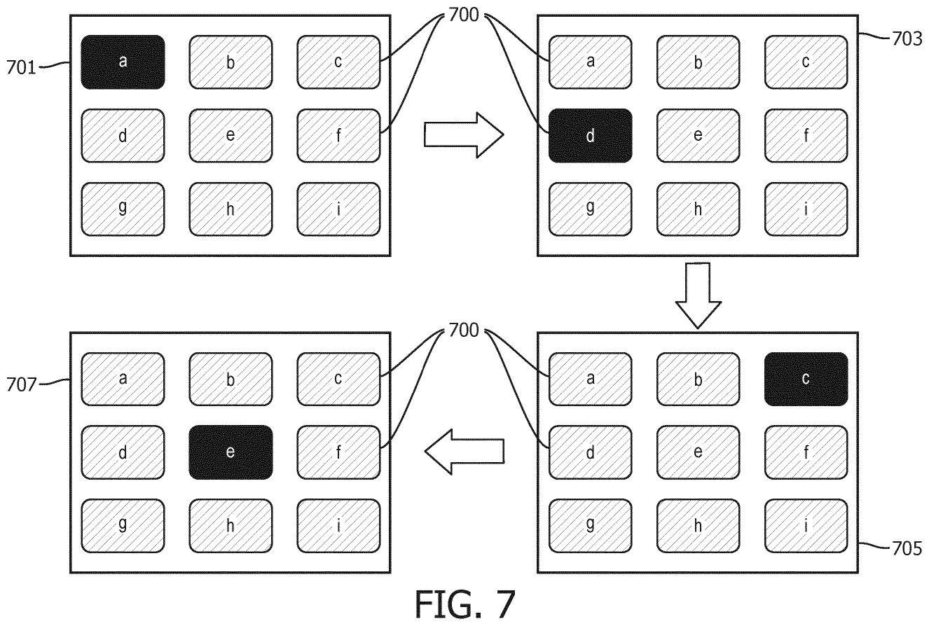

FIG. 7 is an illustration of soft controls provided on a display according to examples of the present disclosure. The soft controls 700 may be provided on a touch screen of an ultrasound imaging system, such as touch screen 310 shown in FIG. 3. The soft controls 700 may also, or alternatively, be provided on a non-touch display, such as display 338 and/or display 138, and a user may interact with the soft controls 700 by manipulating a hard control on a control panel, such as control panel 352 and/or control panel 152. Although all of the soft controls 700 shown in FIG. 7 are buttons, the soft controls 700 may be any combination of soft controls (e.g., buttons, sliders) and any number of soft controls 700 may be provided on the display.

As described with reference to FIG. 6, one or more soft controls 700 may be highlighted (e.g., brighter, different color). In some examples, a soft control 700 may be highlighted based on a prediction of a processor of the ultrasound imaging system (e.g., UI adapter 170) as to the soft control 700 a user will select. The prediction may be based, at least in part, on usage data of the user from earlier user inputs during the exam and/or user inputs from prior exams.

As shown in panel 701, soft control 700a may initially be highlighted. The first soft control 700 to be highlighted may be based, at least in part, on a particular user that has logged in, a selected exam type, or a default function stored in the ultrasound imaging system (e.g., ultrasound imaging system 100 and/or ultrasound imaging system 300). Alternatively, in some examples, no soft control 700 may be highlighted at panel 701.

A user may provide an input to the ultrasound imaging system, such as touching or otherwise manipulating one of the soft controls 700 and/or manipulating a hard control of a control panel (not shown in FIG. 7). In some examples, the user may touch soft control 700a. Responsive, at least in part, to the user's input, a different soft control, such as soft control 700d, may be highlighted as shown in panel 703. The soft control 700d may be highlighted based on a prediction of what the next function the user will select. In some examples, the prediction may be based at least in part on an analysis of usage data. The usage data may include the user input provided in panel 701, and may further include prior usage data (e.g., earlier user inputs during the exam, user inputs from prior exams). In some examples, the processor may change the appearance of soft controls 700 by executing one or more commands.

A user may provide a second input to the ultrasound imaging system, such as touching or otherwise manipulating one of the soft controls 700 and/or manipulating a hard control of a control panel. In some examples, the user may touch soft control 700d, for example, when the processor correctly predicted the next function desired by the user. Responsive, at least in part, to the user's input, the highlighted soft control may be changed by the processor, for example, soft control 700c, as shown in panel 705. Again, soft control 700c may be highlighted based on analysis of usage data. For example, the soft control highlighted in panel 705 may be different depending on whether the user used soft control 700d (e.g., the processor made a correct prediction) or selected a different soft control (e.g., the processor made an incorrect prediction).

A user may provide a third input to the ultrasound imaging system, such as touching or otherwise manipulating one of the soft controls 700 and/or manipulating a hard control of a control panel. In some examples, the user may touch soft control 700c. Responsive, at least in part, to the user's input, the highlighted soft control may be changed by the processor, such as soft control 700e, as shown in panel 707. Again, soft control 700e may be highlighted based on analysis of usage data. For example, the soft control highlighted at panel 707 may be different depending on whether the processor provided correct predictions at panels 703 and 705. Although changes in highlighting of soft controls 700 are shown for three user inputs, the highlighting of soft controls 700 may be altered for any number of user inputs. Furthermore, in some examples, the user inputs provided may be stored for future analysis by the processor for making predictions of the next function desired by the user during a subsequent exam.

By highlighting the soft control most likely to be used next by a user, the user may more quickly locate the desired soft control. Furthermore, in protocol-heavy regions, highlighting the soft control most likely to be used next may help prevent the user from inadvertently skipping a step in the protocol.

Although the examples shown in FIGS. 5-7 are described with reference to soft controls, in applications, these examples may be applied to hard controls. For example, in ultrasound imaging systems with hard controls that are backlit and/or adjacent to light sources in the control panel, the backlighting and/or other light sources may be set to a higher intensity and/or different color to highlight the desired hard controls. Conversely, backlighting and/or other light sources may be dimmed and/or turned off to “fade” less frequently used hard controls. In other words, the illumination of the hard controls may be adapted based on usage data.

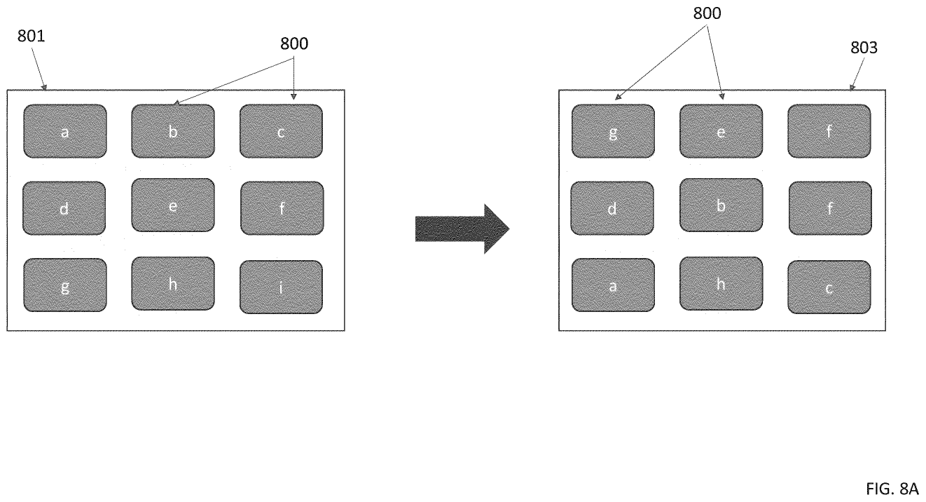

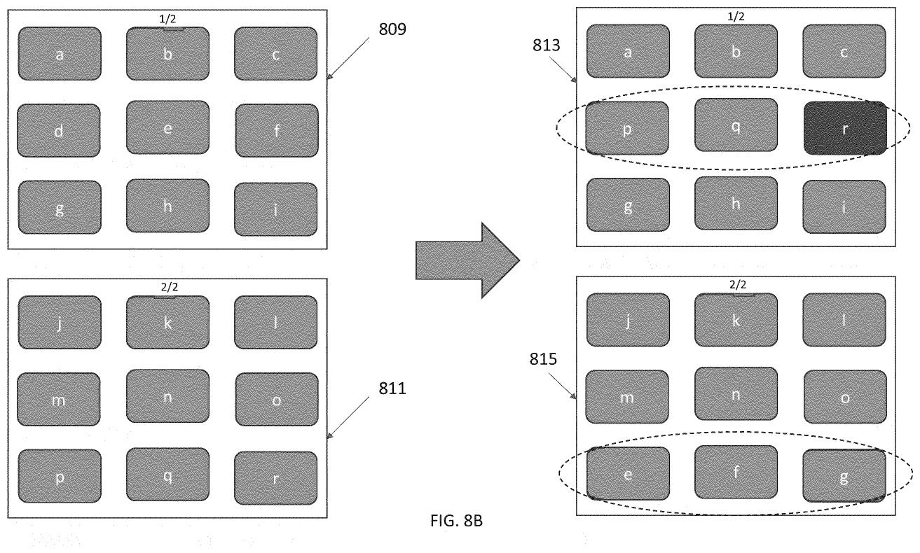

FIG. 8A is an illustration of soft controls provided on a display according to examples of the present disclosure. The soft controls 800 may be provided on a touch screen of an ultrasound imaging system, such as touch screen 310 shown in FIG. 3. The soft controls 800 may also, or alternatively, be provided on a non-touch display, such as display 338 and/or display 138, and a user may interact with the soft controls 800 by manipulating a hard control on a control panel, such as control panel 352 and/or control panel 152. Although all of the soft controls 800 shown in FIG. 8A are buttons, the soft controls 800 may be any combination of soft controls and any number of soft controls 800 may be provided on the display.

In the example shown in FIG. 8A, a processor of the ultrasound imaging system (e.g., UI adapter 170) may analyze usage data to determine which soft controls 800 are used the most by a user. Based on the analysis, the processor may adjust an arrangement of the soft controls 800. In some examples, such as the one shown in panel 801, the soft controls 800 may have an initial arrangement. After a period of use by the user (e.g., one or more user inputs, one or more exams), the arrangement of the soft controls 800 may be altered by the processor. For example, soft controls determined to be most used (e.g., 800g, 800e, and 800f) may be moved further up the display and soft controls determined to be least used (e.g., 800a, 800h, 800c) may be moved lower down the display as shown in panel 803.

In some examples, soft controls 800 may be part of a menu that includes multiple pages as shown by panels 809 and 811 in FIG. 8B. A user may navigate between pages of the menu by “swiping” or manipulating a hard control on the control panel. Multiple pages may be used, for example, when there are more soft controls than can be feasibly shown on a display at the same time. As illustrated by panels 813 and 815, altering the arrangement of the soft controls 800, in addition to repositioning the soft controls 800 on the display, may include altering which page of the menu the soft controls 800 appear. In some examples, lesser used controls (e.g., 800e, 800f, 800g) may be moved from the first page of the menu to the second page and more frequently used controls (e.g., 800p, 800q, 800r) may be moved from the second page to the first page. Although in the example shown in FIG. 8B, the soft controls 800e, 800f, 800g switched places with soft controls 800p, 800q, 800r, adjusting the arrangement of the soft controls 800 on a page of the menu or across pages of the menu, is not limited to this specific example.

By moving more frequently used controls to the top of a display and/or to a first page of a menu, the more frequently used controls may be more visible and easier to find for a user. In some applications, the user may need to spend less time navigating through pages of menus to find a desired control. However, some users may dislike automated rearranging of the soft controls and/or find it disorienting. Accordingly, in some examples, the ultrasound imaging system may allow the user to provide a user input that disables this setting.

Although the example in FIG. 8A is described with reference to soft controls, it may also be applied to hard controls of an ultrasound imaging system. In examples where the ultrasound imaging system includes several reconfigurable hard controls, the most used functions may be assigned to the hard controls in locations most easily accessed by the user whereas lesser used functions may be assigned to more remote locations on the control panel.

In some examples, an ultrasound imaging system may automatically adapt a user interface of the ultrasound imaging system not only based on inputs provided by a user, but also based on what object is being imaged. In some examples, a processor of the ultrasound imaging system, such as image processor 136, may identify anatomy currently being scanned by an ultrasound probe, such as ultrasound probe 112. In some examples, the processor may implement an artificial intelligence/machine learning model trained to identify anatomical features in the ultrasound images. Examples of techniques for identifying anatomical features in ultrasound images may be found in PCT Application PCT/EP2019/084534 filed on Dec. 11, 2019 and entitled “SYSTEMS AND METHODS FOR FRAME INDEXING AND IMAGE REVIEW”. The ultrasound imaging system may adapt the user interface based on the identified anatomical features, for example, by displaying soft controls for functions most commonly used when imaging the identified anatomical features.

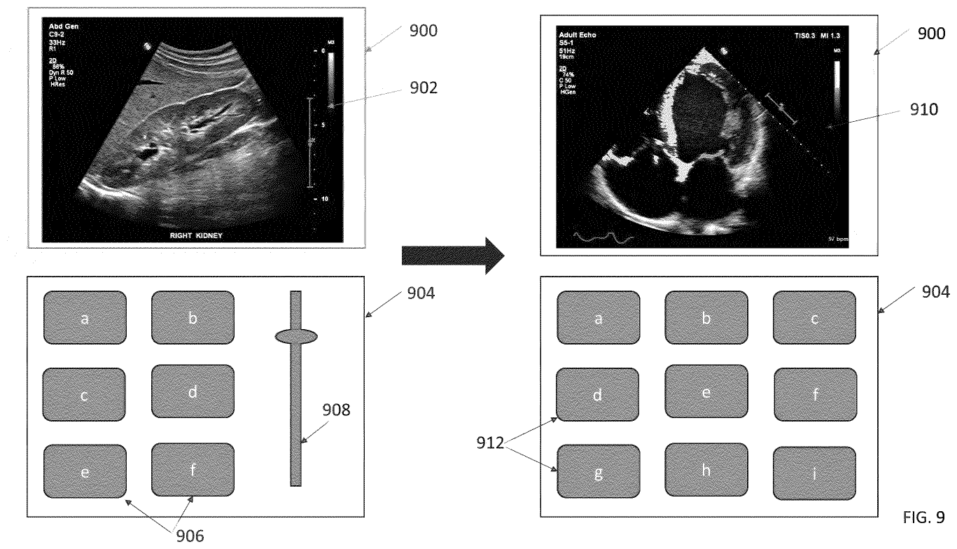

FIG. 9 is an illustration of example ultrasound images on a display and soft controls provided on a display according to examples of the present disclosure. Displays 900 and 904 may be included in an ultrasound imaging system, such as ultrasound imaging system 100 and/or ultrasound imaging system 300. Display 900 may be included in or may be used to implement display 138 and/or display 238 in some examples. Display 904 may be included in or may be used to implement display 138 and/or display 338 in some examples. In some examples, display 904 may be included in or may be used to implement touch screen 310. In some examples, both display 902 and 904 may be touch screens.

Display 900 may provide ultrasound images acquired by an ultrasound probe of the ultrasound imaging system, such as ultrasound probe 112. Display 904 may provide soft controls for manipulation by a user to operate the ultrasound imaging system. However, in other examples (not shown in FIG. 9), both the ultrasound images and the soft controls may be provided on a same display. As noted, the soft controls provided on display 904 may depend, at least in part, on what anatomical feature is being imaged by the ultrasound probe. In the example shown in FIG. 9, on the left-hand side, display 900 is displaying an image of a kidney 902. A processor of the ultrasound imaging system, such as image processor 136, may analyze the image and determine that a kidney is being imaged. This determination may be used to determine what soft controls are provided on display 904. A processor, such as UI adapter 170, may execute commands to alter the soft controls on display 904 based on the determination. In some examples, the same processor may be used to determine what anatomical feature is being imaged as well as adapt the user interface. Continuing the same example, when the ultrasound imaging system recognizes a kidney is being imaged, it may provide buttons 906 and a slider 908. In some examples, buttons 906 and slider 908 may perform functions most commonly used during examination of kidneys. In some examples, the processor may further analyze usage data to determine what soft controls are provided on display 904. Thus, the buttons 906 and slider 908 may perform functions most commonly used by a particular user during examination of kidneys.

On the right-hand side of FIG. 9, display 900 is displaying an image of a heart 910. The processor of the ultrasound imaging system, may analyze the image and determine that a heart is being imaged. This determination may be used to adapt the soft controls provided on display 904. The processor, or another processor, may execute commands to alter the soft controls on display 904 based on the determination. When the ultrasound imaging system recognizes a heart is being imaged, it may provide buttons 912. In some examples, buttons 912 may perform functions most commonly used during echocardiograms. In some examples, the processor may further analyze usage data to determine what soft controls are provided on display 904. Thus, the buttons 912 may perform functions most commonly used by a particular user during echocardiograms.

Although completely different organs, kidney and heart, were shown in the example in FIG. 9, the ultrasound imaging system may be trained to recognize different portions of a same organ or object of interest. For example, it may be trained to recognize different portions of the heart (e.g., left atrium, mitral valve) or different portions of a fetus (e.g., spine, heart, head). The UI may be adapted based on these different portions, not just completely different organs.

Automatic detection of the anatomical features being imaged and dynamically adjusting the user interface may allow a user more efficient access to desired controls. Furthermore, for certain exams, such as fetal scans, different tools may be needed for different portions of the exam, so the UI may not be adequately adapted if based solely on exam type.

Although FIGS. 3-9 are described as separate examples, an ultrasound imaging system may implement more than one and/or combinations of the example UI adaptations described herein. For example, the feature of removing seldom-used controls as described with reference to FIG. 5B may be combined with rearranging controls described with reference with FIGS. 8A-8B such that a two-page menu may be condensed to one page over time. In another example, the examples of FIGS. 3 and 4 may be combined such that both hard and soft controls of a user interface may change function based on a predicted next desired control. In another example, the anatomical feature being imaged may be determined as described with reference to FIG. 9, and the anatomical feature may be used to predict the next control selected by the user as described with reference to FIGS. 3 and 4. In a further example, similar to the example shown in FIG. 9, a set of controls provided on a display to a user may dynamically change, but need not be based on the current anatomical feature being imaged. For example, based on previously selected controls, the processor may predict a next stage of the workflow of the user and provide the controls used in that stage (e.g., after anatomical measurements made on a kidney, may provide controls for Doppler analysis).

Furthermore, other adaptations of the UI that do not directly involve the function, appearance, and/or arrangement of hard and/or soft controls may also be performed based on usage data. For example, a processor may adjust default values of the ultrasound imaging system to create a custom preset based, at least in part on usage data. Examples of default values that may be altered include, but are not limited to, imaging depth, 2D operation, chroma mapping settings, dynamic range, and graymap settings.

According to examples of the present disclosure, ultrasound imaging systems may apply one or more techniques for analyzing usage data to provide automatic adaptations of UIs of ultrasound imaging systems, such as the examples described with reference to FIGS. 3-9. In some examples, the analysis and/or adaptation of the UI may be performed by one or more processors of the ultrasound imaging system (e.g., UI adapter 170).

As disclosed herein, the ultrasound imaging system may receive and store usage data in a computer readable medium, such as local memory 142. Examples of usage data include, but are not limited to, keystrokes, button pushes, other manipulation of hard controls (e.g., turning a dial, flipping a switch), screen touches, other manipulation of soft controls (e.g., swiping, pinching), menu selections and navigation, and voice commands. In some examples, additional usage data may be received such as geographical location of the ultrasound system, type of ultrasound probe used (e.g., type, make, model), unique user identifier, type of exam, and/or what object is currently being imaged by the ultrasound imaging system. In some examples, usage data may be provided by a user via a user interface, such as user interface 124, a processor, such as image processor 136, the ultrasound probe (e.g., ultrasound probe 112), and/or preprogrammed and stored in ultrasound imaging system (e.g., local memory 142).

In some examples, some or all of the usage data may be written to and stored in computer readable files, such as log files, for later retrieval and analysis. In some examples, a log file may store a record of some or all of a user's interactions with the ultrasound imaging system. The log file may include time and/or sequence data such that the time and/or sequence of the different interactions the user had with the ultrasound imaging system may be determined. Time data may include a time stamp that is associated with each interaction (e.g., each keystroke, each button push). In some examples, the log file may store the interactions in a list in the order the interactions occurred such that the sequence of interactions can be determined, even if no time stamp is included in the log file. In some examples, the log file may indicate a particular user that is associated with the interactions recorded in the log file. For example, if a user logs into the ultrasound imaging system with a unique identifier (e.g., username, password), the unique identifier may be stored in the log file. The log file may be a text file, a spreadsheet, a database, and/or any other suitable file or data structure that can be analyzed by one or more processors. In some examples, one or more processors (e.g., UI adapter 170) of the ultrasound imaging system may collect the usage data and write the usage data to one or more log files, which may be stored in the computer readable medium. In some examples, log files and/or other usage data may be received by the imaging system from one or more other imaging systems. The log files and/or other usage data may be stored in the local memory. The log files and/or other usage data may be received by any suitable method, including wireless (e.g., BlueTooth, WiFi) and wired (e.g., Ethernet cable, USB device) methods. Thus, usage data from one or more users as well as from one or more imaging systems may be used for adapting the UI of the imaging system.

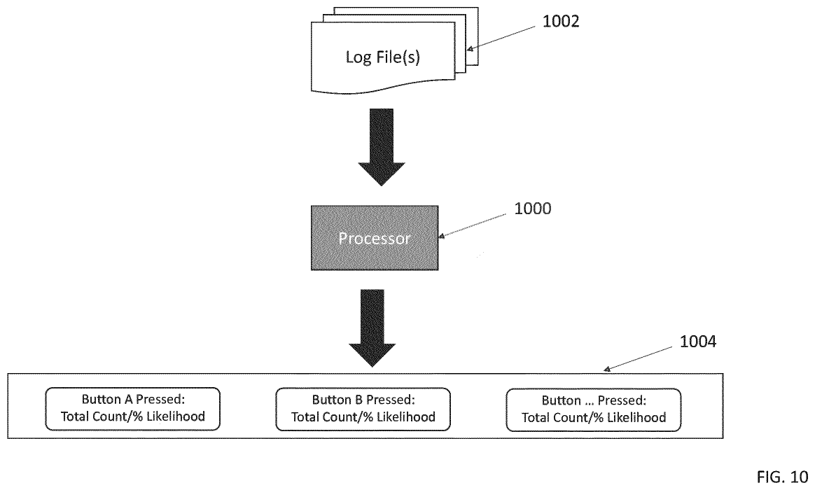

In some examples, the usage data (e.g., such as usage data stored in one or more log files) may be analyzed by statistical methods. A graphical depiction of an example of statistical analysis of one or more log files in accordance with examples of the present disclosure is shown in FIG. 10. A processor 1000 of an ultrasound imaging system (e.g., ultrasound imaging system 100, ultrasound imaging system 300) may receive one or more log files 1002 for analysis. Processor 1000 may be implemented by UI adapter 170 in some examples. The processor 1000 may analyze the usage data in the log files 1002 to calculate various statistics relating to a user interface of an ultrasound imaging system (e.g., user interface 124, user interface 324) to provide one or more outputs 1004. In the specific example shown in FIG. 10, the processor 1000 may determine a total number of times one or more controls (e.g., Button A, Button B, Button C) were selected (e.g., pressed on a control panel and/or touch screen) by one or more users, and the percent likelihood that each of the one or more controls may be selected. In some examples, the percent likelihood may be based on a total number of times a particular control was selected divided by a total number of all control selections.

In some examples, the output 1004 of processor 1000 may be used to adapt a user interface of the ultrasound imaging system (e.g., user interface 124, user interface 324). The user interface may be adapted by processor 1000 and/or another processor of the ultrasound imaging system. For example, the user interface may be adapted such that controls less likely to be selected are faded and/or removed from a display of the user interface as described with reference to FIGS. 5A-5B. In another example, the user interface may be adapted such that controls more likely to be selected are highlighted on the display of the user interface as described with reference to FIG. 6. In another example, controls may be arranged on a display and/or across menus based, at least in part, on their likelihood of being selected as described with reference to FIGS. 8A-8B.

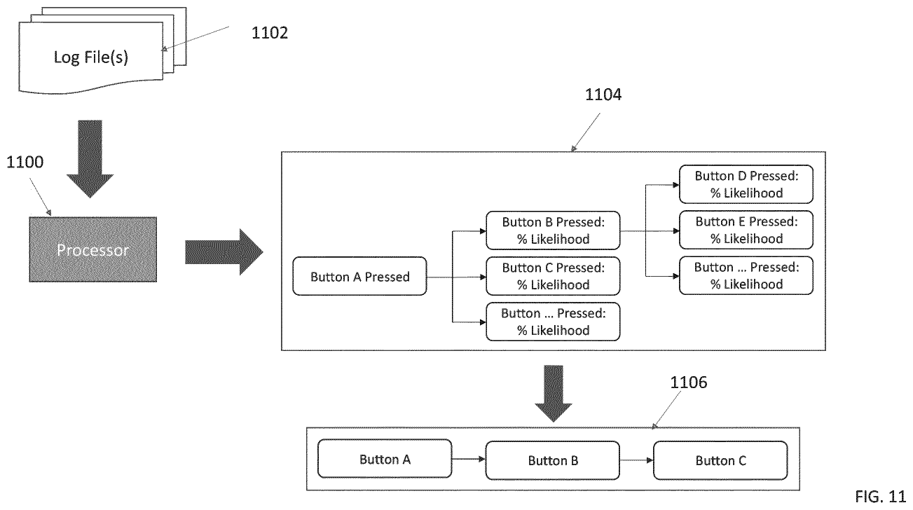

A graphical depiction of another example of statistical analysis of one or more log files in accordance with examples of the present disclosure is shown in FIG. 11. A processor 1100 of an ultrasound imaging system (e.g., ultrasound imaging system 100, ultrasound imaging system 300) may receive one or more log files 1102 for analysis. Processor 1100 may be implemented by UI adapter 170 in some examples. The processor 1100 may analyze the usage data in the log files 1102 to calculate various statistics relating to a user interface of an ultrasound imaging system (e.g., user interface 124, user interface 324) to provide one or more outputs 1104, 1106. As shown in FIG. 11, the processor 1100 may analyze the log files to determine one or more sequences of control selections. The processor 1100 may use a moving window to search for sequences, may search for specific control selections that indicate a start of a sequence (e.g., “start,” “freeze,” “exam type”), and/or other methods (e.g., sequence ends when a time interval between control selections exceeds a maximum duration). For one or more control selections that begin a sequence, the processor 1100 may calculate a percentage likelihood of the next control selected. For example, as shown in output 1104, when Button A is pressed at a beginning of a sequence, the processor 1100 calculates the probability (e.g., percent likelihood) that one or more other controls (e.g., Buttons B, C, etc.) is selected next in the sequence. As shown in output 1104, the processor 1100 may further calculate the probability that one or more other controls (e.g., Button D, Button E, etc.) is selected after one or more of the other controls selected after Button A. This calculation of probabilities may continue for any desired sequence length.

Based on the output 1104, the processor 1100 may calculate a most likely sequence of control selections by a user. As shown in output 1106, it may be determined that Button B has the highest probability of being selected by a user after Button A is selected by the user and Button C has the highest probability of being selected by the user after Button B has been selected by the user.

In some examples, the output 1106 of processor 1100 may be used to adapt a user interface of the ultrasound imaging system (e.g., user interface 124, user interface 324). The user interface may be adapted by processor 1100 and/or another processor of the ultrasound imaging system. For example, the user interface may be adapted such that a function of a hard or a soft control may be changed to the most likely desired function as described with reference to FIGS. 3 and 4. In another example, the user interface may be adapted such that a control that is most likely desired function is highlighted on a display as described with reference to FIG. 7.

The analysis of log files, including the examples of statistical analysis described with reference to FIGS. 10 and 11, may be performed as usage data is being received and recorded (e.g., live capture) to the log files and/or analysis may be performed at a later time (e.g., a pause in a workflow, end of an exam, logoff of the user).

While statistical analysis of log files have been described, in some examples, one or more processors (e.g., UI adapter 170) of an ultrasound imaging system may implement one or more trained artificial intelligence, machine learning, and/or deep learning models (collectively referred to as AI models) for analyzing usage data whether in log files or other formats (e.g., live capture prior to storing in a log file). Examples of models that may be used to analyze usage data include, but are not limited to, decision trees, convolutional neural networks, and long short term memory (LSTM) networks. In some examples, using one or more AI models may allow for faster and/or more accurate analysis of usage data and/or faster adaptation of a user interface of the ultrasound imaging system responsive to the usage data. More accurate analysis of usage data may include, but are not limited to, more accurate predictions of a next selected control in a sequence, more accurate predictions of the most likely used controls for a particular user during a particular exam type, and/or more accurate determination of an anatomical feature being imaged.

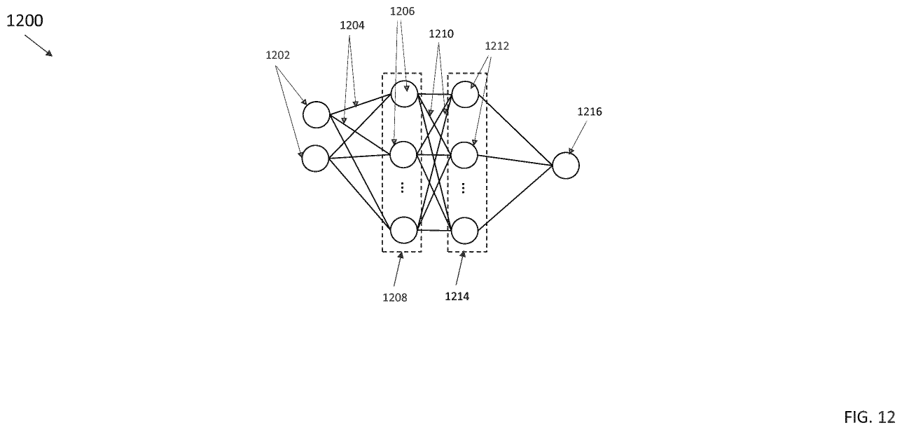

FIG. 12 is an illustration of a neural network that may be used to analyze usage data in accordance with examples of the present disclosure. In some examples, the neural network 1200 may be implemented by one or more processors (e.g., UI adapter 170, image processor 136) of an ultrasound imaging system (e.g., ultrasound imaging system 100, ultrasound imaging system 300). In some examples, neural network 1200 may be a convolutional network with single and/or multidimensional layers. The neural network 1200 may include one or more input nodes 1202. In some examples, the input nodes 1202 may be organized in a layer of the neural network 1200. The input nodes 1202 may be coupled to one or more layers 1208 of hidden units 1206 by weights 1204. In some examples, the hidden units 1206 may perform operations on one or more inputs from the input nodes 1202 based, at least in part, with the associated weights 1204. In some examples, the hidden units 1206 may be coupled to one or more layers 1214 of hidden units 1212 by weights 1210. The hidden units 1212 may perform operations on one or more outputs from the hidden units 1206 based, at least in part, on the weights 1210. The outputs of the hidden units 1212 may be provided to an output node 1216 to provide an output (e.g., inference) of the neural network 1200. Although one output node 1216 is shown in FIG. 12, in some examples, the neural network may have multiple output nodes 1216. In some examples, the output may be accompanied by a confidence level. The confidence level may be a value from, and including, 0 to 1, where a confidence level 0 indicates the neural network 1200 has no confidence that the output is correct and a confidence level of 1 indicates the neural network 1200 is 100% confident that the output is correct.

In some examples, inputs to the neural network 1200 provided at the one or more input nodes 1202 may include log files, live capture usage data, and/or images acquired by an ultrasound probe. In some examples, outputs provided at output node 1216 may include a prediction of a next control selected in a sequence, a prediction of controls likely to be used by a particular user, controls likely to be used during a particular exam type, and/or controls likely to be used when a particular anatomical feature is being imaged. In some examples, outputs provided at output node 1216 may include a determination of an anatomical image currently being imaged by an ultrasound probe (e.g., ultrasound probe 112) of the ultrasound imaging system.

The outputs of neural network 1200 may be used by an ultrasound imaging system to adapt (e.g., adjust) a user interface of the ultrasound imaging system (e.g., user interface 124, user interface 324). In some examples, the neural network 1200 may be implemented by one or more processors of the ultrasound imaging system (e.g., UI adapter 170, image processor 136). In some examples, the one or more processors of the ultrasound imaging system (e.g., UI adapter 170) may receive an inference of the controls most used (e.g., manipulated, selected) by a user. Based on the inference, the processor may fade and/or remove lesser used controls (e.g., as described with reference to FIGS. 5A-B) or highlight more frequently used controls (e.g., as described with reference to FIG. 6). In some examples, the processor may move more likely used controls to a top of a display and/or to a first page of a multi-page menu as described with reference to FIGS. 8A-8B.

In some examples, the processor may receive multiple outputs from neural network 1200 and/or multiple neural networks that may be used to adapt the user interface of the ultrasound imaging system. For example, the processor may receive an output indicating an anatomical feature currently being imaged by an ultrasound probe (e.g., ultrasound probe 112) of the ultrasound imaging system. The processor may also receive an output indicating controls most typically used by a user when the particular anatomical feature is imaged. Based on these outputs, the processor may execute commands to provide the most typically used controls on a display as described with reference to FIG. 9.

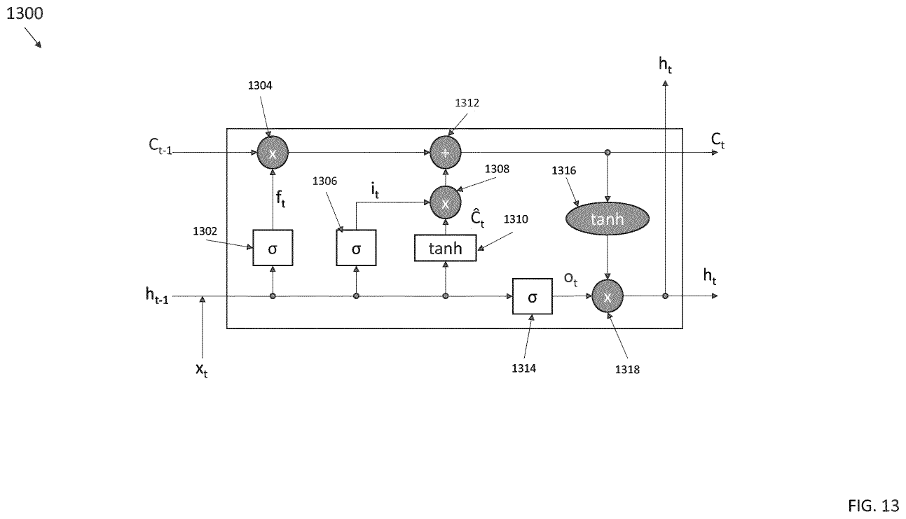

FIG. 13 is an illustration of a cell of a long short term memory (LSTM) model that may be used to analyze usage data in accordance with examples of the present disclosure. In some examples, the LSTM model may be implemented by one or more processors (e.g., UI adapter 170, image processor 136) of an ultrasound imaging system (e.g., ultrasound imaging system 100, ultrasound imaging system 300). A LSTM model is a type of recurrent neural network that is capable learning long-term dependencies. Accordingly, LSTM models may be suitable for analyzing and predicting sequences, such as sequences of user selections of various controls of a user interface of an ultrasound machine. An LSTM model typically includes multiple cells coupled together. The number of cells may be based, at least in part, on a length of a sequence to be analyzed by the LSTM. For simplicity, only a single cell 1300 is shown in FIG. 13.

The variable C, running across the top of cell 1300 is the state of the cell. The state of the previous LSTM cell Cₜ₋₁ may be provided to cell 1300 as an input. Data can be selectively added or removed from the state of the cell by cell 1300. The addition or removal of data is controlled by three “gates,” each of which includes a separate neural network layer. The modified or unmodified state of cell 1300 may be provided by cell 1300 to the next LSTM cell as Cₜ.

The variable h, running across the bottom of the cell 1300 is the hidden state vector of the LSTM model. The hidden state vector of the previous cell hₜ₋₁ may be provided to cell 1300 as an input. The hidden state vector hₜ₋₁ may be modified by a current input xₜ to the LSTM model provided to cell 1300. The hidden state vector may also be modified based on the state of the cell 1300 Cₜ. The modified hidden state vector of cell 1300 may be provided as an output hₜ. The output hₜ may be provided to the next LSTM cell as a hidden state vector and/or provided as an output of the LSTM model.

Turning now to the inner workings of cell 1300, a first gate (e.g., the forget gate) for controlling a state of the cell C includes a first layer 1302. In some examples, this first layer is a sigmoid layer. The sigmoid layer may receive a concatenation of the hidden state vector hₜ₋₁ and the current input xₜ. The first layer 1302 provides an output fₜ, which includes weights that indicate which data from the previous cell state should be “forgotten” and which data from the previous cell state should be “remembered” by cell 1300. The previous cell state Cₜ₋₁ is multiplied by fₜ at point operation 1304 to remove any data that was determined to be forgotten by the first layer 1302.

A second gate (e.g., the input gate) includes a second layer 1306 and a third layer 1310. Both the second layer 1306 and the third layer 1310 receive the concatenation of the hidden state vector hₜ₋ₜ and the current input xₜ. In some examples, the second layer 1306 is a sigmoid function. The second layer 1306 provides an output iₜ which includes weights that indicate what data needs to be added to the cell state C. The third layer 1310 may include a tanh function in some examples. The third layer 1310 may generate a vector Ĉₜ that includes all possible data that can be added to the cell state from hₜ₋ₜ and xₜ. The weights iₜ and vector Cₜ are multiplied together by point operation 1308 generates a vector that includes the data to be added to the cell state C. The data is added to the cell state C to get the current cell state Cₜ at point operation 1312.

A third gate (e.g., the output gate) includes a fourth layer 1314. In some examples, the fourth layer 1314 is a sigmoid function. The fourth layer 1314 receives the concatenation of the hidden state vector hₜ₋₁ and the current input xₜ and provides an output oₜ which includes weights that indicate what data of the cell state Cₜ should be provided as the hidden state vector hₜ of cell 1300. The data of the cell state Cₜ is turned into a vector by a tanh function at point operation 1316 and is then multiplied by oₜ by point operation 1318 to generate the hidden state vector/output vector hₜ. In some examples, the output vector hₜ may be accompanied by a confidence value, similar to the output of a convolutional neural network, such as the one described in reference to FIG. 12.

As pictured in FIG. 13, cell 1300 is a “middle” cell. That is, the cell 1300 receives inputs Cₜ₋₁ and hₜ₋₁ from a previous cell in the LSTM model and provides outputs Cₜ and hₜ to a next cell in the LSTM. If cell 1300 were a first cell in the LSTM, it would only receive input xₜ. If cell 1300 were a last cell in the LSTM, the outputs hₜ and Cₜ would not be provided to another cell.

In some examples where a processor of an ultrasound imaging system (e.g., UI adapter 170) implements an LSTM model, the current input xₜ may include data related to a control selected by a user and/or other usage data. The hidden state vector hₜ₋₁ may include data related to a previous prediction of a control selection by a user. The cell state Cₜ₋₁ may include data related to previous selections made by the user. In some examples, output(s) hₜ of the LSTM model may be used by the processor and/or another processor of the ultrasound imaging system to adapt a user interface of the ultrasound imaging system (e.g., user interface 124, user interface 324). For example, when hₜ includes predictions of a next control selected by a user, the processor may use the prediction to alter a function of a hard or a soft control as described with reference to FIGS. 3 and 4. In another example, the processor may use the prediction to highlight a soft control on a display as described with reference to FIG. 7.

As described herein, the AI/machine learning models (e.g., neural network 1200 and LSTM including cell 1300) may provide confidence levels associated with one or more outputs. In some examples, a processor (e.g., UI adapter 170) may only adapt a UI of an ultrasound imaging system if the confidence level associated with the output is equal to or above a threshold value (e.g., over 50%, over 70%, over 90%, etc.). In some examples, if the confidence level is below the threshold value, the processor may not adapt the UI. In some examples, this may mean not fading, highlighting, removing, switching, and/or rearranging controls on a display. In some examples, this may mean not changing a function of a hard or soft control (e.g., maintaining the existing function).

Although a convolutional neural network and a LSTM model have been described herein, these AI/machine learning models have been provided only as examples, and the principles of the present disclosure are not limited to these particular models.

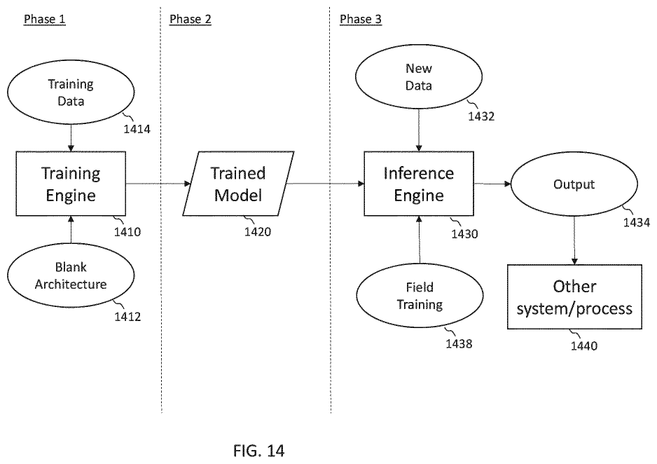

FIG. 14 shows a block diagram of a process for training and deployment of a model in accordance with the principles of the present disclosure. The process shown in FIG. 14 may be used to train a model (e.g., artificial intelligence algorithm, neural network) included in an ultrasound system, for example, a model implemented by a processor of the ultrasound system (e.g., UI adapter 170) The left hand side of FIG. 14, phase 1, illustrates the training of a model. To train the model, training sets which include multiple instances of input arrays and output classifications may be presented to the training algorithm(s) of the model(s) (e.g., AlexNet training algorithm, as described by Krizhevsky, A., Sutskever, I. and Hinton, G. E. “ImageNet Classification with Deep Convolutional Neural Networks,” NIPS 2012 or its descendants). Training may involve the selection of a starting algorithm and/or network architecture 1412 and the preparation of training data 1414. The starting architecture 1412 may be a blank architecture (e.g., an architecture with defined layers and arrangement of nodes but without any previously trained weights, a defined algorithm with or without a set number of regression coefficients) or a partially trained model, such as the inception networks, which may then be further tailored for analysis of ultrasound data. The starting architecture 1412 (e.g., blank weights) and training data 1414 are provided to a training engine 1410 for training the model. Upon sufficient number of iterations (e.g., when the model performs consistently within an acceptable error), the model 320 is said to be trained and ready for deployment, which is illustrated in the middle of FIG. 14, phase 2. The right hand side of FIG. 14, or phase 3, the trained model 1420 is applied (via inference engine 1430) for analysis of new data 1432, which is data that has not been presented to the model during the initial training (in phase 1). For example, the new data 1432 may include unknown data such as live keystrokes acquired from a control panel during a scan of a patient (e.g., during an echocardiography exam). The trained model 1420 implemented via engine 1430 is used to analyze the unknown data in accordance with the training of the model 1420 to provide an output 1434 (e.g., least used buttons on a display, next likely input, anatomical feature being imaged, confidence level). The output 1434 may then be used by the system for subsequent processes 1440 (e.g., fade a button on a display, change a functionality of a hard control, highlight a button on a display).

In the examples where the trained model 1420 is used as a model implemented or embodied by a processor of the ultrasound system (e.g., UI adapter 170), the starting architecture may be that of a convolutional neural network, a deep convolutional neural network, or a long short term memory model in some examples, which may be trained to determine least or most used controls, predict a next likely control selected, and/or determine an anatomical feature being imaged. The training data 1414 may include multiple (hundreds, often thousands or even more) annotated/labeled log files, images, and/or other recorded usage data. It will be understood that the training data need not include a full image or log file produced by an imaging system (e.g., a log file representative of every user input during an exam, an image representative of the full field of view of an ultrasound probe) but may include patches or portions of log files or images. In various examples, the trained model(s) may be implemented, at least in part, in a computer-readable medium comprising executable instructions executed by a processor or processors of an ultrasound system, e.g., UI adapter 170.

As described herein, an ultrasound imaging system may automatically and/or dynamically change a user interface of the ultrasound imaging system based, at least in part, on usage data from one or more users. However, in some examples, the ultrasound imaging system may allow a user to adjust the user interface. Allowing the user to adjust the user interface may be in addition to or instead of automatically and/or dynamically changing the user interface by the ultrasound imaging system (e.g., by one or more processors, such as the UI adapter 170).

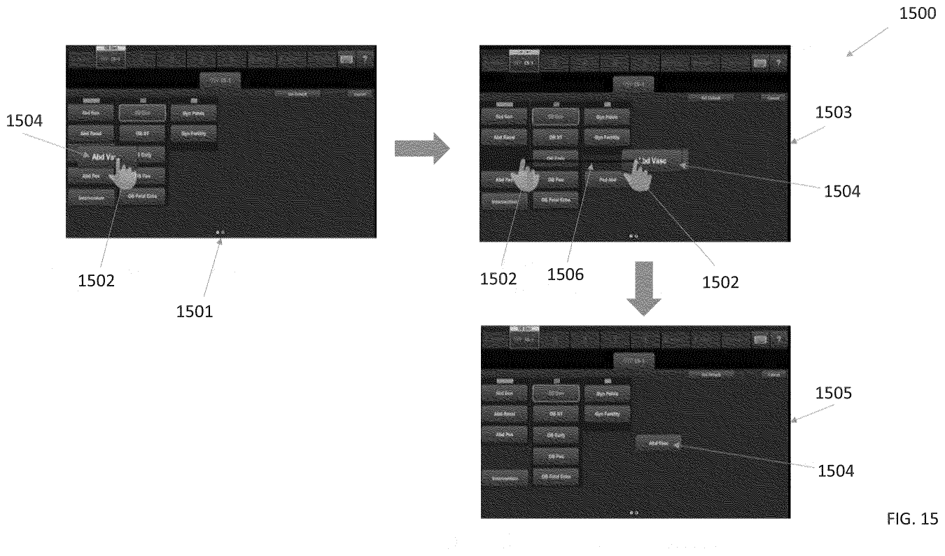

FIGS. 15-19 illustrate examples of how a user of an ultrasound imaging system (e.g., ultrasound imaging system 100, ultrasound imaging system 300) may adjust a user interface (e.g., user interface 124, user interface 324) of the ultrasound imaging system. In some examples, the user may provide user inputs for adjusting the user interface. The inputs may be provided via a control panel (e.g., control panel 152, control panel 352), which may or may not include a touch screen (e.g., touch screen 310). In examples with a touch screen, the user may provide inputs by pressing, tapping, dragging, and/or other gestures. In examples without a touch screen, the user may provide inputs via one or more hard controls (e.g., buttons, dials, sliders, switches, trackball, mouse, etc.). Responsive to the user inputs, one or more processors (e.g., UI adapter 170, graphics processor 140) may adapt the user interface. The examples provided with reference to FIGS. 15-19 are for illustrative purposes only and the principles of the present disclosure are not limited to these particular ways a user may adapt the user interface of the ultrasound imaging system.

FIG. 15 shows a graphical overview of a user moving a button within a page of a menu provided on a display in accordance with examples of the present disclosure. In some examples, the menu may be provided on display 138, display 338, and/or touch screen 310. As shown in panel 1501, a user 1502 may press and hold a button 1504. In some examples, the user 1502 may press and hold a finger on a touch screen (e.g., touch screen 310) where button 1504 is displayed. In some examples, the user 1502 may move a cursor over the button 1504 and press and hold a button on a control panel (e.g., control panel 152, control panel 352). After a delay, the button 1504 may “pop out of its original location and “snap” to the location of the user's 1502 finger and/or cursor. As shown in panel 1503, the user 1502 may drag the button 1504 to a new location as shown by line 1506 by dragging the finger across the touch screen or moving the cursor while still depressing the button on the control panel. The button 1504 may follow the user's 1502 finger and/or cursor. The user 1502 may move the finger away from the touch screen or release the button on the control panel and the button 1504 may “snap” into the new location as shown in panel 1505.

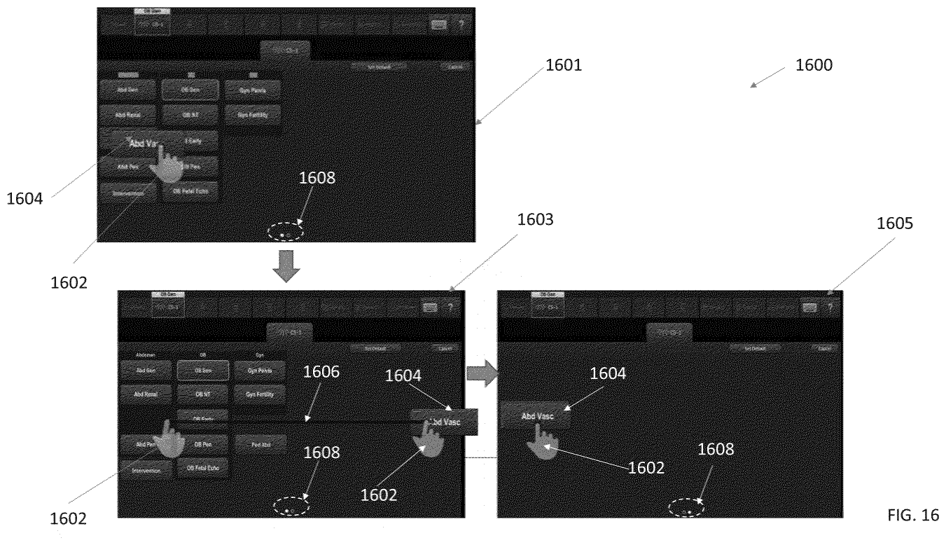

FIG. 16 shows a graphical overview of a user moving a button between pages of a menu on a display in accordance with examples of the present disclosure. In some examples, the menu may be provided on display 138, display 338, and/or touch screen 310. As shown in panel 1601, a user 1602 may press and hold a button 1604. In some examples, the user 1602 may press and hold a finger on a touch screen (e.g., touch screen 310) where button 1604 is displayed. In some examples, the user 1602 may move a cursor over the button 1604 and press and hold a button on a control panel (e.g., control panel 152, control panel 352). After a delay, the button 1604 may “pop out of its original location and “snap” to the location of the user's 1602 finger and/or cursor. As discussed herein, for example, with reference to FIG. 8B, menu 1600 may have multiple pages as indicated by dots 1608. In some examples, such as the one in FIG. 16, the dot that is colored in may indicate the current page of menu 1600 that is displayed. The user 1602 may drag the button 1604 to an edge of the menu as shown by line 1606 by dragging the finger across the touch screen or moving the cursor while still depressing the button on the control panel. When the user 1602 reaches a vicinity of the edge of the screen, as shown in panel 1603, the menu 1600 may automatically navigate (e.g., display) to a next page (the second page in this example) of menu as shown in panel 1605. The user 1602 may move the finger away from the touch screen or release the button on the control panel and the button 1604 may “snap” into the new location on the second page.

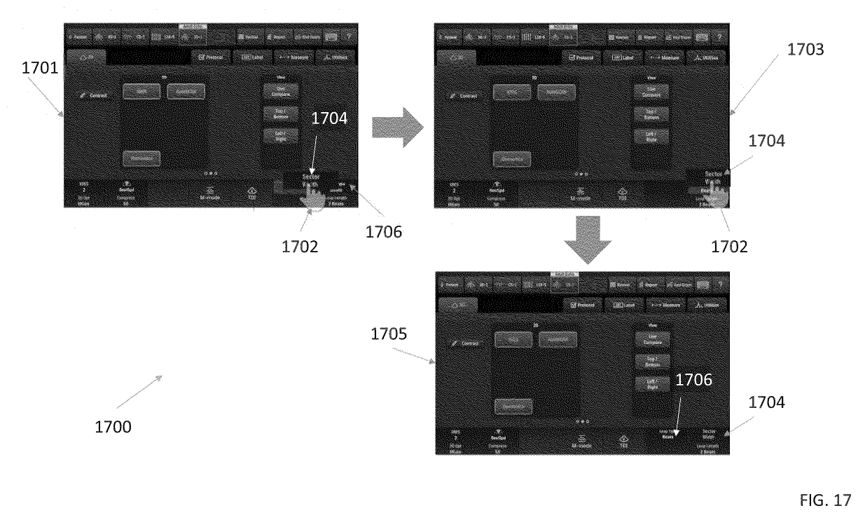

FIG. 17 shows a graphical overview of a user swapping locations of buttons on a display in accordance with examples of the present disclosure. In some examples, the menu may be provided on display 138, display 338, and/or touch screen 310. As shown in panel 1701, a user 1702 may press and hold a button 1704. In some examples, the user 1702 may press and hold a finger on a touch screen (e.g., touch screen 310) where button 1704 is displayed. In some examples, the user 1702 may move a cursor over the button 1704 and press and hold a button on a control panel (e.g., control panel 152, control panel 352). After a delay, the button 1704 may “pop out of its original location and “snap” to the location of the user's 1702 finger and/or cursor. The user 1702 may drag the button 1704 to a desired location over another button 1706 by dragging the finger across the touch screen or moving the cursor while still depressing the button on the control panel as shown in panel 1703. As shown in panel 1705, the user 1702 may move the finger away from the touch screen or release the button on the control panel and the button 1704 may “snap” into the location of button 1706 and button 1706 may switch to the original location of button 1704.

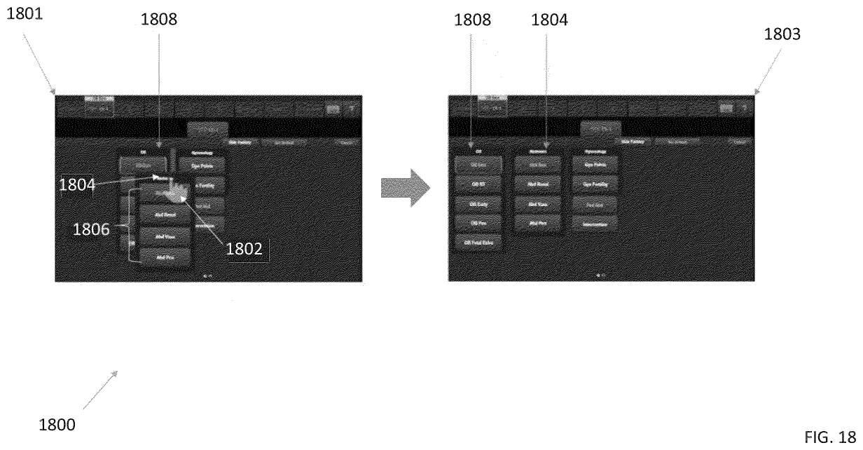

FIG. 18 shows a graphical overview of a user moving a group of buttons on a display in accordance with examples of the present disclosure. In some examples, the menu may be provided on display 138, display 338, and/or touch screen 310. As shown in FIG. 18, in some examples, one or more buttons 1806 may be organized into a group, such as groups 1804 and 1808. As shown in panel 1801, a user 1802 may press and hold a heading of group 1804. In some examples, the user 1802 may press and hold a finger on a touch screen (e.g., touch screen 310) where group 1804 is displayed. In some examples, the user 1802 may move a cursor over the heading of group 1804 and press and hold a button on a control panel (e.g., control panel 152, control panel 352). After a delay, the group 1804 may “pop out of its original location and “snap” to the location of the user's 1802 finger and/or cursor. The user 1802 may drag the group 1804 to a new location by dragging the finger across the touch screen or moving the cursor while still depressing the button on the control panel. The group 1804 may follow the user's 1802 finger and/or cursor. The user 1802 may move the finger away from the touch screen or release the button on the control panel and the group 1804 may “snap” into the new location as shown in panel 1803. If a group, such as group 1808 was already present in the desired location, the group 1808 may move to the original location of group 1804.

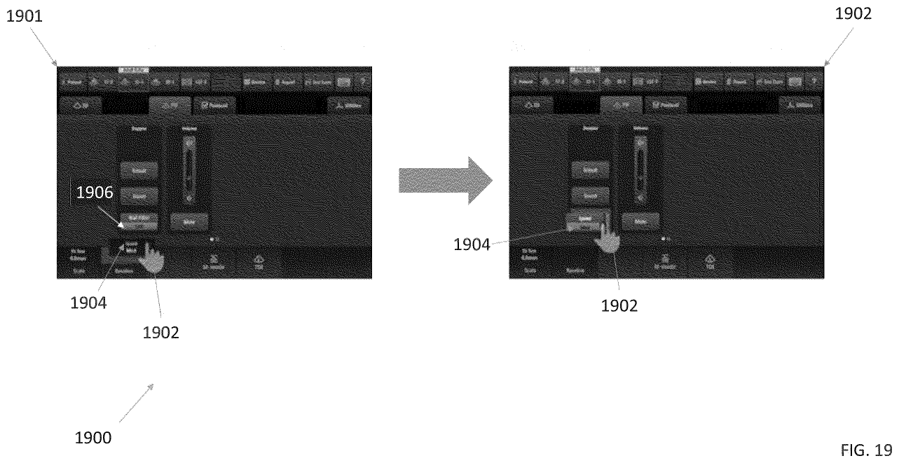

FIG. 19 shows a graphical overview of a user changing a rotary control to a list button on a display in accordance with examples of the present disclosure. In some examples, the menu may be provided on display 138, display 338, and/or touch screen 310. In some examples, some buttons, such as button 1904, may be a rotary control, while others, such as button 1906 may be a list button. As shown in panel 1901, a user 1902 may press and hold button 1904. In some examples, the user 1902 may press and hold a finger on a touch screen (e.g., touch screen 310) where button 1904 is displayed. In some examples, the user 1902 may move a cursor over the button 1904 and press and hold a button on a control panel (e.g., control panel 152, control panel 352). After a delay, the button 1904 may “pop out of its original location and “snap” to the location of the user's 1902 finger and/or cursor. As shown in panel 1903, the user 1902 may drag the button 1904 to a new location in a list by dragging the finger across the touch screen or moving the cursor while still depressing the button on the control panel. The button 1904 may follow the user's 1902 finger and/or cursor. The user 1902 may move the finger away from the touch screen or release the button on the control panel and the button 1904 may “snap” into the new location. In some examples, if another button, such as button 1906 is in the desired location for button 1904, button 1906 may replace button 1904 as a rotary control. In other examples, button 1906 may shift up or down in the list to accommodate button 1904 becoming a list button.

As disclosed herein, an ultrasound imaging system may include a user interface that may be customized by a user. Additionally, or alternatively, the ultrasound imaging system may automatically adapt the user interface based on usage data of one or more users. The ultrasound imaging systems disclosed herein may provide a customized, adaptable UI for each user. In some applications, automatically adapting the UI may reduce exam time, improve efficiency, and/or provide ergonomic benefits to the user.

In various embodiments where components, systems and/or methods are implemented using a programmable device, such as a computer-based system or programmable logic, it should be appreciated that the above-described systems and methods can be implemented using any of various known or later developed programming languages, such as “C”, “C++”, “C#”, “Java”, “Python”, and the like. Accordingly, various storage media, such as magnetic computer disks, optical disks, electronic memories and the like, can be prepared that can contain information that can direct a device, such as a computer, to implement the above-described systems and/or methods. Once an appropriate device has access to the information and programs contained on the storage media, the storage media can provide the information and programs to the device, thus enabling the device to perform functions of the systems and/or methods described herein. For example, if a computer disk containing appropriate materials, such as a source file, an object file, an executable file or the like, were provided to a computer, the computer could receive the information, appropriately configure itself and perform the functions of the various systems and methods outlined in the diagrams and flowcharts above to implement the various functions. That is, the computer could receive various portions of information from the disk relating to different elements of the above-described systems and/or methods, implement the individual systems and/or methods and coordinate the functions of the individual systems and/or methods described above.

In view of this disclosure it is noted that the various methods and devices described herein can be implemented in hardware, software and firmware. Further, the various methods and parameters are included by way of example only and not in any limiting sense. In view of this disclosure, those of ordinary skill in the art can implement the present teachings in determining their own techniques and needed equipment to affect these techniques, while remaining within the scope of the invention. The functionality of one or more of the processors described herein may be incorporated into a fewer number or a single processing unit (e.g., a CPU) and may be implemented using application specific integrated circuits (ASICs) or general purpose processing circuits which are programmed responsive to executable instruction to perform the functions described herein.

Although the present system may have been described with particular reference to an ultrasound imaging system, it is also envisioned that the present system can be extended to other medical imaging systems where one or more images are obtained in a systematic manner. Accordingly, the present system may be used to obtain and/or record image information related to, but not limited to renal, testicular, breast, ovarian, uterine, thyroid, hepatic, lung, musculoskeletal, splenic, cardiac, arterial and vascular systems, as well as other imaging applications related to ultrasound-guided interventions. Further, the present system may also include one or more programs which may be used with conventional imaging systems so that they may provide features and advantages of the present system. Certain additional advantages and features of this disclosure may be apparent to those skilled in the art upon studying the disclosure, or may be experienced by persons employing the novel system and method of the present disclosure. Another advantage of the present systems and method may be that conventional medical image systems can be easily upgraded to incorporate the features and advantages of the present systems, devices, and methods.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1. A medical imaging system comprising:
a user interface comprising a plurality of controls, each of the plurality of controls configured to be manipulated by a user for changing an operation of the medical imaging system;
a memory configured to store usage data resulting from the manipulation of the plurality of controls; and
a processor in communication with the user interface and the memory, wherein the processor is configured to:
receive the usage data;
determine, based on the usage data, a first control of the plurality of controls associated with lower frequency of usage than a second control of the plurality of controls; and
adapt the user interface based on the frequency of usage by reducing a visibility of the first control, increasing the visibility of the second control, or a combination thereof.

2. The medical imaging system of claim 1, wherein the usage data comprises a plurality of log files, each associated with a different imaging session, and wherein the processor is configured to further reduce or increase the visibility of one or more of the plurality of controls based on the frequency of usage determined from the plurality of log files.

3. The medical imaging system of claim 1, wherein the first control and the second control each comprise a respective hard control and an illumination associated with the respective hard control, and wherein the processor is configured to reduce and increase the visibility of the first and second controls, respectively by reducing and increasing the illumination associated with the respective mechanical control.

4. The medical imaging system of claim 1, wherein the user interface comprises a display and the plurality of controls are soft controls provided on the display.

5. The medical imaging system of claim 4, wherein reducing the visibility of the first control comprises dimming backlighting of the display at a location of the first control on the display, increasing a translucency of a graphical user interface element corresponding to the first control, or a combination thereof.

6. The medical imaging system of claim 4, wherein increasing the visibility of the second control comprises increasing a brightness or changing a color of the second control.

7. The medical imaging system of claim 2, wherein the processor is configured to incrementally reduce the visibility of at least one of the soft controls over time and remove the at least one soft control from the display when the frequency of usage falls below a predetermined threshold.

8. The medical imaging system of claim 1, wherein the frequency of usage is determined using statistical analysis.

9. The medical imaging system of claim 1, wherein the determining, based on the usage data, comprises determining, for individual ones of the plurality of controls, a number of times a given control of the plurality of controls is selected, and comparing the number of times the given control is selected to a total number of times all of the plurality of controls are selected to determine the frequency of usage of the given control.

10. A medical imaging system comprising:
a user interface comprising a plurality of controls configured to be manipulated by a user for changing an operation of the medical imaging system;
a memory configured to store usage data resulting from the manipulation of the plurality of controls; and
a processor in communication with the user interface and the memory, the processor configured to:
receive the usage data;
receive an indication of a first selected control of the plurality of controls, wherein the first selected control is associated with a first function;
determine, based at least in part on the usage data and the first function, a next predicted function; and
following manipulation of the first control, adapt the user interface by changing the function of one of the plurality of controls to the next predicted function, increasing a visibility of the control configured to perform the next predicted function relative to other controls of the plurality of controls, or a combination thereof.

11. The medical imaging system of claim 10, wherein the processor is configured, following the manipulation of the first control, to change the function of the first control to the next predicted function.

12. The medical imaging system of claim 10, wherein the user interface comprises a control panel, and wherein the processor is configured to change the function of one of a plurality of hard controls provided on the control panel to the next predicted function or to increase the visibility of the hard control that is associated with the next predicted function relative to other hard controls on the control panel.

13. The medical imaging system of claim 10, wherein the processor implements an artificial intelligence model to analyze the usage data and determine one or more sequences of control selections.

14. The medical imaging system of claim 13, wherein the artificial intelligence model includes a long short term memory model.

15. The medical imaging system of claim 13, wherein the artificial intelligence model further outputs a confidence level associated with the next predicted function, and wherein the processor is configured to adapt the user interface only when the confidence level is equal to or above a threshold value.

16. The medical imaging system of claim 10, wherein increasing the visibility of the control configured to perform the next predicted function relative to other controls of the plurality of controls comprises at least one of increasing a brightness or changing a color of the control configured to perform the next predicted function.

17. The medical imaging system of claim 10, wherein the usage data further comprises a make and a model of the ultrasound probe, a user identifier, a geographical location of the ultrasound imaging system, or a combination thereof.

18. The medical imaging system of claim 10, further comprising an ultrasound probe configured to acquire ultrasound signals for generating an ultrasound image, wherein the processor is further configured to determine an anatomical feature included in the ultrasound image and the next predicted function is based, at least in part, on the anatomical feature.

19. The medical imaging system of claim 18, wherein the processor implements a convolutional neural network to analyze the ultrasound image and determine the anatomical feature included in the ultrasound image.

20. The medical imaging system of claim 18, further comprising a second display for displaying the ultrasound image.
